(19) United States
(12) Patent Application Publication
(10) Pub. No.: US 2025/0134811 A1
(43) Pub. Date: May 1, 2025

(54) ULTRAFLEXIBLE LIPOSOMES IN GEL FORMULATION

(71) Applicant: The Research Foundation for The State University of New York, Binghamton, NY (US)

(72) Inventors: Anthony Di Pasqua, Rochester, NY (US); Mengwei Sun, Vestal, NY (US)

(21) Appl. No.: 18/835,345

(22) PCT Filed: Jan. 12, 2023
(86) PCT No.: PCT/US2023/060596

§ 371 Date: Aug. 1, 2024

Related U.S. Application Data
(60) Provisional application No. 63/299,158, filed on Jan. 13, 2022.

(51) Int. Cl. A61K 9/127; A61K 9/00; A61K 9/06; A61K 31/167; A61K 47/20; A61K 47/32; A61P 23/02

(52) U.S. Cl. CPC A61K 9/127; A61K 9/0014; A61K 9/06; A61K 31/167; A61K 47/20; A61K 47/32; A61P 23/02

(57) ABSTRACT

Ultraflexible liposomes are formed using soy phosphatidylcholine, lidocaine, and different amount of sodium cholate, a surfactant. The prepared ultraflexible liposomes (UFLs) were formulated into a gel using carbomer as a gelling agent. The topical gel loaded with ultraflexible liposomal lidocaine shows enhanced skin permeation effect along with an increase in local anesthetic action of lidocaine.

### FIELD OF THE INVENTION

The present invention relates to liposomal delivery systems for pharmaceutical agents, and more particularly to ultraflexible liposomes in a gel for transdermal drug delivery, e.g., lidocaine.

### BACKGROUND OF THE INVENTION

Liposomes contain three distinct environments for drugs to dissolve in: the water-lipid interface, the hydrophobic core, and the aqueous interior. As such, they are useful to dissolve/carry hydrophobic, hydrophilic and amphiphilic drugs and antioxidants. This is important since many drugs of therapeutical interest have poor water solubilities and/or form unstable solutions. Pandit, Jayamanti, Minakshi Garg, and Narendra Kumar Jain. “Miconazole nitrate bearing ultraflexible liposomes for the treatment of fungal infection.” Journal of liposome research 24, no. 2 (2014): 163-169. Further, liposomes may be useful in transporting drugs across impermeable or lipophilic barriers. See also Arciniegas, S. M., M. J. Bernad, S. C. Caballero, D. Hern, B. Solis, and D. Vargas. “Process for physicochemical improvement of ultra-flexible liposomes loaded with insulin.” Indian Journal of Pharmaceutical Sciences 81, no. 1 (2019): 110-116.

A variety of drug delivery systems have been developed to enhance skin penetration such as microparticles, ethosomes, solid lipid nanoparticles, and ultraflexible liposomes (UFLs). Among them, microparticles show bigger particle size and poor skin permeation than solid lipid nanoparticles and UFLs; and ethosomes lack long-term structural and chemical stability during storage, in addition to possible skin irritation caused by high ethanol content. UFLs are biocompatible bilayer vesicular carriers that can deliver numerous drugs for therapeutic, biochemical, and cosmetic purposes. They are also known as deformable liposomes, ultradeformable liposomes, flexible liposomes, clastic liposomes, and transfersomes (pioneered by IDEA AG, Munich, Germany). UFLs are capable of traversing intact across the skin layer by creating a water content difference in the upper layer of skin and inner viable epidermis, which generates transepidermal hydration gradients. This hydration gradients contribute to successful stratum corneum crossing of UFLs by diffusion-mediated mechanisms. It has also been mentioned that the negative charge of these liposomal nanoparticles is favorable for transdermal permeation. The electrostatic repulsion generated between UFLs and intercellular components of skin causes accelerated penetration of UFLs through follicles of different skin layers.

In the field of topical vehicles for dermal/transdermal delivery, ultra-flexible liposomes are often considered the vehicle of choice, mainly because of their high performance as transdermal penetration enhancers and their good stability in suspension.

The main drawback of the UFLs when used topically is their liquidity, making it difficult for localized application. To solve this issue, these nanoliposomes may be incorporated into an appropriate vehicle to protect the intrinsic structure of the vesicles.

Carbomer has been reported to sustain drug release and show good bioadhesion and has been used together with soybean phosphatidylcholine.

Ultra-flexible liposomes are composed by a mixture of lipids with low phase transition temperatures and an appropriate amount of a detergent. The detergent acts as a membrane destabilizer, producing an increase in membrane deformability. Ultra-flexible liposomes high deformability has been proposed to be the cause of their ability to penetrate the skin and even allow for proteins to reach systemic circulation.

Attempts have been made to deliver lidocaine using UFL technology. M E Planas, P. González, L., Rodríguez, S., Sánchez, and G. Cevc, “Percutaneous, non-invasive induction of topical analgesia through a novel type of drug carriers and the prolongation of local pain insensitivity by analgesic liposomes”, Anesth., Analg. (1992), 95:615-621 report the results of investigations with formulations containing surfactant, typically loaded with lidocaine (2%, as a free base) in a suspension at 4-8% of mixed lipids (w/v). Lipid aggregates from a 4:1 mol/mol mixture of phosphatidylcholine/sodium cholate were prepared, starting with a solution of ethanolic lipids (7-3% by weight of EtOH in the final product) for easier production. All tested suspensions were unstable.

The topical or transdermal route of drug delivery has numerous advantages over other routes because of patient compliance, preventing the first-pass effect or pre-systemic metabolism of drugs by the liver, with few side effects. However, the major issue with these systems is the transport of therapeutic moieties across the skin due to the tiny pore size of 20-40 nm, which acts as a barrier. Topical dermatological dosage forms—such as creams. lotions, and novel carrier-based topical formulations—can be directed to treat skin diseases. By administrating drug molecules to the precise site of action, such as locations of skin diseases, topical formulation products provide the quickest and handiest treatment technique for eczema, psoriasis, wound dressings, oncology-related diseases, and anal fissures. Sudhakar, Kalvatala, Shivkanya Fuloria, Vetriselvan Subramaniyan, Kathiresan V. Sathasivam, Abul Kalam Azad, Shasank S. Swain, Mahendran Sekar et al. “Ultraflexible Liposome Nanocargo as a Dermal and Transdermal Drug Delivery System.” Nanomaterials 11, no. 10 (2021): 2557.

Solid lipid nanoparticles (SLNs) are nanostructured lipid carriers (NLCs) that fall in the category of rigid lipid carriers and liposome vesicles, while liquid micelles (LMs) are ultraflexible systems. Vesicular carriers—such as transfersomes, ethosomes, and transethosomes—are the new generation of ultraflexible carriers. SLNs are colloidal carriers, and the purpose of developing these carriers is to overcome the problems related to traditional carriers (such as emulsions, liposomes, and polymeric nanoparticles). SLNs, which are hydrophobic, are exposed to phagocytic uptake by macrophages and targeted by lymph capillaries and lymph nodes, which serve as reservoirs for viruses and other microorganisms. SLNs have gained considerable attention as novel colloidal drug carriers for intravenous applications rather than for transdermal drug delivery.

Nanotechnology in dermatology has dramatically improved traditional liposome compositions in achieving the deeper permeation of active ingredients to different skin strata. Liposomes of ultraflexible vesicles are common vectors in transdermal drug delivery systems that are relatively liquid and deformed. Currently, there are three types of highly flexible liposomes: transfersomes, ethosomes, and transethosomes. In recent years, ultraflexible vesicles have become new liposome carriers with high deformability, high trapping efficiency, a reasonable transdermal drug delivery permeation rate, and suitable transdermal administration.

Transfersomes are drug transporters that can permeate intact deep skin. Unimpeded passage of these cargo carriers may be predicated by two factors: the high elasticity (ultraflexible or deformability) of the bilayer vesicle, and an osmotic gradient around the skin. Transfersomes have high surface hydrophilicity, and respond to the gradient of hydration across the dermal tissue. Transfersome vesicles have a high degree of flexibility in the bilayer membrane. The mechanism behind transfersomes' penetration is the development of the osmotic gradient generated due to the evaporation of the lipid suspension on the skin's surface as water evaporates. Transfersomes are strong, intensely deformed bilayers and, therefore, have an increased ability to bind and retain water. Dehydration does not occur in an ultradeformable and highly hydrophilic vesicle; it is not the same as forward osmosis, but it may be related to forward osmosis in the transport process.

Ethosomes represent the third generation of elastic lipid vesicular carriers, containing phospholipids, ethanol, and water. Ethosomes have been reported to improve the supply of different medications to the skin. Ethosomal systems differ from liposomes because the ethanol content of their formulations is relatively high. Ethosomal systems are classified into three classes based on their compositions: classical ethosomes, binary ethosomes, and transethosomes (TEs). Song et al. first reported transethosomes in 2012, and the transethosomal system framework contains the fundamental parts of the traditional ethosome, with extra ingredients—for example, permeation enhancers or surface-active agents in their lipid carrier formulation. Transethosomes made of phospholipids, ethanol, water, and an edge activator (surface-active agent) or permeation enhancer have been created. The amalgamation of transfersomes (ultradeformable vesicles) and ethosomes (elastic and flexible vesicles) can cause the vesicular delivery of the drug to go deeper into the skin's tissue.

The pain caused by lidocaine injections in the face prior to facial plastic surgeries to remove growths or tumorous lesions has been reported by many patients to be the worst part of these procedures. However, lidocaine gels and creams currently on the market do not allow for an equal or better local anesthetic effect to replace these injections. To enhance the penetration of lidocaine through the skin, an effective delivery vehicle is urgently needed. In the field of topical vehicles for dermal/transdermal delivery, ultraflexible liposomes (UFL) are often considered the vehicle of choice, mainly due to their superior performance in transdermal penetration and their good stability in suspension. UFL are usually formulated by a mixture of lipids with low phase transition temperatures and an appropriate amount of detergent. As a membrane destabilizer, the detergent enables UFL to be highly clastic and deformable. Thus, they can escape from narrow pores in the stratum corneum (one-tenth of their own diameter) under nonocclusive conditions.

The main drawback of the liposomes when used topically is their liquidity, making it difficult for localized application. To overcome this, liposomes are incorporated into a suitable vehicle in which the original structure of the vesicles is preserved. It has been a well-known fact that the liposomes are compatible with the gel systems made from the polymers such as carbomer. Carbomer may be used as a gelling agent for the incorporation of the UF and traditional liposomes for the topical delivery system because of its low potential for skin irritation and sensitization.

The primary mechanism of action of lidocaine as a local anesthetic is via blocking the voltage-gated sodium channels which induces a reversible block of action potential propagation. Currently, lidocaine is mainly administered via subcutaneous, intramuscular, or intravenous injection, but this conventional method of delivery has many inherent limitations in clinical application. Anesthesia used in surgical procedures related with superficial skin, such as skin transplantation, skin lesion removal, esthetic surgeries, tattooing, birthmark removal, and scar revision, usually demand numerous injections. Multiple rounds of needle injection as well as the invasive nature of injections not only require administration by trained personnel, but also elicit pain and discomfort, resulting in lower acceptance/compliance by patients. Also, the generation of sharp contaminants poses safety problems.

Rationally, many issues involving injection could potentially be solved by advanced drug delivery methodologies such as transdermal drug delivery. However, approaches for transdermal delivery usually require a long onset time. For example, the onset time for EMLA™ cream, a commercially-available topical anesthetic containing 2.5% lidocaine and 2.5% prilocaine is 60 min, which might interfere with the efficiency of the surgery. Besides, some dosage forms such as pastes, creams, and ointment for topical drug delivery can be easily removed by wetting, movement, and contact. Thus, developing a biocompatible, bioadhesive, and efficient transdermal delivery vehicle with short onset time and prolonged anesthesia duration, is beneficial to both patients and physicians.

Poly(acrylic acid) (PAA; trade name Carbomer) is a polymer with the formula (CH₂—CHCO₂H)ₙ. It is a derivative of acrylic acid (CH₂═CHCO₂H). In addition to the homopolymers, a variety of copolymers and crosslinked polymers, and partially deprotonated derivatives thereof are known. In a water solution at neutral pH, PAA is an anionic polymer, i.e.., many of the side chains of PAA lose their protons and acquire a negative charge. Partially or wholly deprotonated PAAs are polyelectrolytes, with the ability to absorb and retain water and swell to many times their original volume. These properties—acid-base and water-attracting—are the bases of many applications.

Polyacrylic acid is a polyolefin. It can be viewed as polyethylene with carboxylic acid (CO₂H) substituents on alternating carbons. Owing to these groups, alternating carbon atoms in the backbone are stereogenic (colloquially: chiral). For this reason, acrylic acid exists in atactic, syndiotactic, and isotactic forms, although this aspect is rarely discussed. The polymerization is initiated with radicals and is assumed to be stereorandom. Crosslinking can be introduced in many ways.

Polyacrylic acid is a weak anionic polyelectrolyte, whose degree of ionization is dependent on solution pH. In its non-ionized form at low pHs, PAA may associate with various non-ionic polymers (such as polyethylene oxide, poly-N-vinyl pyrrolidone, polyacrylamide, and some cellulose ethers) and form hydrogen-bonded interpolymer complexes. In aqueous solutions PAA can also form polycomplexes with oppositely charged polymers such as chitosan, surfactants, and drug molecules (for example, streptomycin).

The neutralized polyacrylic acid gels are suitable biocompatible matrices for medical applications such as gels for skin care products. PAA films can be deposited on orthopedic implants to protect them from corrosion. Crosslinked hydrogels of AA and gelatin have also been used as medical glue. Hydrogels derived from PAA have attracted much study for use as bandages and aids for wound healing.

Gels are semisolid dosage forms that contain an agent (a gelling agent) to provide stiffness to a solution or a colloidal dispersion. Gels do not flow at low shear stress and generally exhibit plastic flow behavior. The gel compositions of the present invention could be hydrogels.

Polyacrylic acid (or carboxypolymethylene polymers, or polyacrylates, or acrylic acid polymers), are well known in the pharmaceutical, cosmetic and food industry. More particularly, these polymers are widely used in the pharmaceutical industry as dispersing, emulsifying, suspending or thickening agents. Such polymers are available from Noveon, Inc (Cleveland, Ohio, USA), under the trademarks Carbopol®, Pemulen®, Noveon® Polycarbophil. The USP-NF, European Pharmacopoeia, British Pharmacopoeia, United States Adopted Names Council (USAN), and International Nomenclature for Cosmetic Ingredients (INCI) have adopted the generic (i.e., non-proprietary) name “carbomer” for various homopolymer polymers. The Japanese Pharmaceutical Excipients list carbomer homopolymers as “carboxyvinyl polymer” and “carboxy polymethylene.” The Italian Pharmacopoeia also identifies Carbopol 934P as “carboxy polymethylene” and the Deutschen Artzneibuch calls Carbopol 980NF “polyacrylic acid.” Carbopol copolymers, such as Carbopol 1342 NF and 1382, and the Pemulen® polymeric emulsifiers, have also been named “carbomer” by the USP-NF, but are considered “Acrylates/C10-C30 Alkyl Acrylates Crosspolymer” by the INCI. The Noveon® series of products is generically known as “polycarbophil”. All of these polymers have the same acrylic acid backbone. The main differences are related to presence of comonomer and crosslink density. Specifically, the polymers are either homopolymers of acrylic acid cross-linked with allyl sucrose or allyl pentaerythritol (Carbopol® homopolymers); homopolymers of acrylic acid cross-linked with divinyl glycol (Noveon® polycarbophils); or copolymers of acrylic acid with minor levels of long chain alkyl acrylate comonomers crosslinked with allylpentaerythritol (Carbopol® copolymers and Pemulen® polymeric emulsifiers). The molecular weight of these polymers is theoretically estimated to range from 700,000 to 3 or 4 billion. All these polymers are herein designated as carbomers. In most liquid systems, carbomers require neutralization to thicken most efficiently. Sodium hydroxide, potassium hydroxide, ammonium hydroxide, and some water-soluble organic amines are excellent neutralizing agents for carbomers in water systems. In all cases the solution viscosity increases as the various carbomers are neutralized.

A flat plateau is reached for the pH range of 5 to 10 and a loss in efficiency occurs as higher pH is obtained. Apart from neutralization with bases, carbomer dispersions can also be thickened by another mechanism, called hydrogen-bonding. Some commonly used hydroxyl donors arc: polyols (such as glycerin, propylene glycol and polyethylene glycols), sugar alcohols such as mannitol, nonionic surfactants with five or more ethoxy groups, glycol-silane copolymers, polyethylene oxide, and fully hydrolyzed polyvinyl alcohol, among others. These reagents hydrogen-bond with the polymer molecule causing it to uncoil: sec Noveon's Technical Data Sheet 43, “Carbopol® polymers can thicken without neutralization”, January 2002. Amino acids are also useful as neutralizing agents for Carbopol® polymers: see Noveon's Technical Data Sheet 53, “Amino Acid Salts of Carbopol® Polymers”, January 2002. The man of the art is kindly asked to refer to Noveon's Products Specifications, Pharmaceutical Bulletins and Technical Data Sheets for further description and information on carbomer polymers of interest in the present invention.

In the context of pharmaceutical active agents, it is known that the presence of carboxy groups in carbomers makes them ionic and permits of the formation of salts such as metal salts, and other complexes. Sec US 2008/0138391.

Whether or not a system behaves as a gel (i.e., exhibits semisolid characteristics, rather than acting as a liquid or solid, etc.) will depend on the various components used within the system and the relative ratios of the different components. It may also depend on the method by which the components that make up the system are mixed, e.g., the order in which the various components are introduced to each other. It is therefore possible for an agent to act as a gelling agent in one environment but not in another. The ability to test compositions to confirm that they are gels as defined herein is within the knowledge of the skilled person in view of the present disclosure and common general knowledge in the field.

The viscosity of a gel can depend on temperature. At low temperatures (e.g., 2-8° C.) the viscosity can be relatively high, but after applying a gel composition of the invention to the skin it can become less viscous because of the combination of increased temperature and the physical stress while being applied. This shear-thinning characteristic gives a gel which is easily distributed on the skin.

In order to effect formation of a gel, it is necessary to have an agent in the composition which acts as a gelling agent. The amount of the gelling agent (or gelling agents, in embodiments where two or more gelling agents are used) required to form a gel will vary on the components within the particular composition. It is common (although not required) to select two or more components which, when used together in particular amounts, effect formation of a gel. These components would typically include an emulsifier and/or viscosity-increasing ingredient with an aqueous buffer solution.

In general, gels are non-invasive and have a localized effect with minimum side effects. Gel compositions of the invention should be suitable for human topical administration. Thus the compositions have the appropriate physical characteristics of topical gels. For instance, the gel compositions have good spreadability, i.e., the gels can readily be spread (e.g., using fingers) after application to the skin to provide a uniform layer. The gel compositions also have excellent extrudability. These properties mean that the gel compositions of the invention are particularly suitable for topical administration. In some embodiments, the gel compositions are applied topically and do not leave a visible residue. The volatile components of the gel compositions may also substantially evaporate to dryness after a certain period of time following topical application. Preferably, the volatile components of the gel composition will evaporate after a therapeutically effective amount of the ingenol-3-angelate has penetrated into the skin (e.g., after about 1 minute, 2 minutes, 5 minutes, 10 minutes, 20 minutes, etc. following topical administration to a subject).

A number of clinical trials have been conducted on liposomal lidocaine:

ClinicalTrials.gov (Other terms = “liposomal lidocaine”)

| | | Study | | | |
|---|---|---|---|---|---|
| Title | Status | Results | Conditions | Interventions | Locations |
| The Effect of | Completed | Has | Pain | Drug: | |
| Liposomal Lidocaine | | Results | | Liposomal | |
| on Perceived Pain in | | | | Lidocaine | |
| Children During | | | | Drug: Placebo | |
| Percutaneous | | | | | |
| Interosseous Pin | | | | | |
| Removal in the | | | | | |
| Outpatient Setting | | | | | |
| A Comparison of | Withdrawn | No | Pain | Drug: Ametop | Children's |
| Amethocaine Cream | | Results | | (4% | Hospital of |
| vs. Liposomal | | Available | | Amethocaine | Western Ontario, |
| Lidocaine Cream for | | | | cream) | London, Ontario, |
| Venipuncture in | | | | Drug: Maxilene | Canada |
| Children. | | | | (4% Liposomal | |
| | | | | Lidocaine) | |
| Evaluation of Topical | Completed | No | Pain | Drug: | Mount Sinai |
| Liposomal Lidocaine | | Results | | Lidocaine | Hospital, Toronto, |
| and Oral Sucrose for | | Available | | Drug: 24% | Ontario, Canada |
| Treatment of Pain in | | | | Sucrose | The Hospital for |
| Newborns | | | | Solution | Sick Children, |
| Undergoing | | | | | Toronto, Ontario, |
| Venipuncture | | | | | Canada |
| Liposomal Lidocaine | Completed | No | Anesthesia | Drug: | Piracicaba Dental |
| Gel for Oral Topical | | Results | | Lidocaine | School, |
| Anesthesia | | Available | | | Piracicaba, São |
| | | | | | Paulo, Brazil |
| Comparison of | Completed | No | Pain | Drug: Synera | Women and |
| Synera Patch Versus | | Results | Venipuncture | Patch | Children Hospital, |
| LMX-4 Cream | | Available | Anesthetics, | Drug: LMX 4 | Charleston, West |
| Versus Placebo Patch | | | Local | Cream | Virginia, United |
| for Pain Reduction | | | | Drug: Placebo | States |
| During Venipuncture | | | | Patch | |
| in Children | | | | | |
| Randomized Control | Completed | Has | Pain | Drug: 4% | Lehigh Valley |
| Trial of a Topical | | Results | Anxiety | lidocaine | Hospital, |
| Anesthetic to | | | | topical | Allentown, |
| Evaluate Pain and | | | | anesthetic | Pennsylvania, |
| Anxiety During | | | | cream | United States |
| Venipuncture | | | | Drug: | |
| | | | | Placebo cream | |
| Comparison of | Completed | No | Pain | Drug: | The Hospital for |
| Different Methods | | Results | Anxiety | Liposomal | Sick Children, |
| for Reducing Pain | | Available | | lidocaine | Toronto, Ontario, |
| From Influenza | | | | Drug: | Canada |
| Vaccine Injections | | | | Vapocoolant | |
| | | | | spray | |
| | | | | Other: Rubbing | |
| | | | | adjacent to the | |
| | | | | injection site | |
| | | | | Other: | |
| | | | | Distraction | |
| Study of Functional | Withdrawn | No | Wounds | Device: | |
| Microarray- | | Results | | functional | |
| Facilitated Lidocaine | | Available | | microarray | |
| Liposomal Cream | | | | | |
| Absorption for | | | | | |
| Cutaneous | | | | | |
| Anesthesia in | | | | | |
| Volunteers | | | | | |
| Lumbar Punctures in | Terminated | Has | Meningitis | Drug: 1% | MedStar |
| Neonates: Improving | | Results | | lidocaine | Georgetown |
| Success Rates and | | | | Drug: topical | University |
| Minimizing Pain | | | | lidocaine | Hospital, |
| | | | | | Washington, |
| | | | | | District of |
| | | | | | Columbia, United |
| | | | | | States |

### SUMMARY OF THE INVENTION

The present technology provides a gel containing ultraflexible liposomes comprising a phospholipid, a surfactant, and an aqueous phase containing a drug to be delivered. (In the case of lipophilic drugs or amphoteric drugs, the active agent may be contained in the lipid phase in both phases, or at an interface layer.) The amount of surfactant needed for ultraflexible liposomal gel preparation to achieve the highest penetration ability was optimized. The formulated gel was characterized to show the stability and viscosity profiles of the formulated gel. The analgesic effect was demonstrated through tail-flick test in rats.

Carbomer is a preferred gelling agent for the incorporation of the UFLs to develop topical delivery systems because of its low potential for skin irritation and sensitization, good bioadhesive property, and good thermal stability.

The lipid bilayer (or phospholipid bilayer) is a thin polar membrane made of two layers of lipid molecules. These membranes are flat sheets that form a continuous barrier around all cells. The cell membranes of almost all organisms and many viruses are made of a lipid bilayer, as are the nuclear membrane surrounding the cell nucleus, and membranes of the membrane-bound organelles in the cell. The lipid bilayer is the barrier that keeps ions, proteins and other molecules where they are needed and prevents them from diffusing into areas where they should not be. Lipid bilayers are ideally suited to this role, even though they are only a few nanometers in width, because they are impermeable to most water-soluble (hydrophilic) molecules. Bilayers are particularly impermeable to ions, which allows cells to regulate salt concentrations and pH by transporting ions across their membranes using proteins called ion pumps. en.wikipedia.org/wiki/Lipid_bilayer

Biological bilayers are usually composed of amphiphilic phospholipids that have a hydrophilic phosphate head and a hydrophobic tail consisting of two fatty acid chains. Phospholipids with certain head groups can alter the surface chemistry of a bilayer and can, for example, serve as signals as well as “anchors” for other molecules in the membranes of cells. Just like the heads, the tails of lipids can also affect membrane properties, for instance by determining the phase of the bilayer. The bilayer can adopt a solid gel phase state at lower temperatures but undergo phase transition to a fluid state at higher temperatures, and the chemical properties of the lipids' tails influence at which temperature this happens. The packing of lipids within the bilayer also affects its mechanical properties, including its resistance to stretching and bending. Many of these properties have been studied with the use of artificial “model” bilayers produced in a lab. Vesicles made by model bilayers have also been used clinically to deliver drugs

Biological membranes typically include several types of molecules other than phospholipids. A particularly important example in animal cells is cholesterol, which helps strengthen the bilayer and decrease its permeability.

Cholesterol also helps regulate the activity of certain integral membrane proteins. Integral membrane proteins function when incorporated into a lipid bilayer, and they are held tightly to the lipid bilayer with the help of an annular lipid shell.

There are three distinct regions of a typical lipid bilayer: the fully hydrated headgroups, the fully dehydrated alkane core, and a short intermediate region with partial hydration. Although the head groups are neutral, they have significant dipole moments that influence the molecular arrangement. The lipid bilayer is very thin compared to its lateral dimensions.

The first region on either side of the bilayer is the hydrophilic headgroup. This portion of the membrane is completely hydrated and is typically around 0.8-0.9 nm thick. In phospholipid bilayers the phosphate group is located within this hydrated region, approximately 0.5 nm outside the hydrophobic core. In some cases, the hydrated region can extend much further, for instance in lipids with a large protein or long sugar chain grafted to the head. One common example of such a modification in nature is the lipopolysaccharide coat on a bacterial outer membrane, which helps retain a water layer around the bacterium to prevent dehydration.

Next to the hydrated region is an intermediate region that is only partially hydrated. This boundary layer is approximately 0.3 nm thick. Within this short distance, the water concentration drops from 2M on the headgroup side to nearly zero on the tail (core) side. The hydrophobic core of the bilayer is typically 3-4 nm thick, but this value varies with chain length and chemistry. Core thickness also varies significantly with temperature, in particular near a phase transition.

In many naturally occurring bilayers, the compositions of the inner and outer membrane leaflets are different. In human red blood cells, the inner (cytoplasmic) leaflet is composed mostly of phosphatidylethanolamine, phosphatidylserine and phosphatidylinositol and its phosphorylated derivatives. By contrast, the outer (extracellular) leaflet is based on phosphatidylcholine, sphingomyelin and a variety of glycolipids. In some cases, this asymmetry is based on where the lipids are made in the cell and reflects their initial orientation.

When a cell undergoes apoptosis, the phosphatidylserine, normally localized to the cytoplasmic leaflet, is transferred to the outer surface: There, it is recognized by a macrophage that then actively scavenges the dying cell. Therefore, a liposome with phosphatidyl serine on its outer layer may have high uptake into a macrophage.

At a given temperature a lipid bilayer can exist in either a liquid or a gel (solid) phase. All lipids have a characteristic temperature (Curie temperature) at which they transition (melt) from the gel to liquid phase. In both phases the lipid molecules are prevented from flip-flopping across the bilayer, but in liquid phase bilayers a given lipid will exchange locations with its neighbor millions of times a second. This random walk exchange allows lipid to diffuse and thus wander across the surface of the membrane. Unlike liquid phase bilayers, the lipids in a gel phase bilayer have less mobility. The presence of impurities, such as cholesterol and cholic acid, disrupts membrane organization and increases fluidity at a given temperature.

The phase behavior of lipid bilayers is determined largely by the strength of the attractive Van der Waals interactions between adjacent lipid molecules. Longer-tailed lipids have more area over which to interact, increasing the strength of this interaction and, as a consequence, decreasing the lipid mobility. Thus, at a given temperature, a short-tailed lipid will be more fluid than an otherwise identical long-tailed lipid. Transition temperature can also be affected by the degree of unsaturation of the lipid tails. An unsaturated double bond can produce a kink in the alkane chain, disrupting the lipid packing. This disruption creates extra free space within the bilayer that allows additional flexibility in the adjacent chains.

Most natural membranes are a complex mixture of different lipid molecules. If some of the components are liquid at a given temperature while others are in the gel phase, the two phases can coexist in spatially separated regions, rather like an iceberg floating in the ocean. This phase separation plays a critical role in biochemical phenomena because membrane components such as proteins can partition into one or the other phase and thus be locally concentrated or activated. One particularly important component of many mixed phase systems is cholesterol, which modulates bilayer permeability, mechanical strength, and biochemical interactions.

While lipid tails primarily modulate bilayer phase behavior, it is the headgroup that determines the bilayer surface chemistry. Most natural bilayers are composed primarily of phospholipids, but sphingolipids and sterols such as cholesterol are also important components. Of the phospholipids, the most common headgroup is phosphatidylcholine (PC), accounting for about half the phospholipids in most mammalian cells. PC is a zwitterionic headgroup, as it has a negative charge on the phosphate group and a positive charge on the amine but, because these local charges balance, no net charge. Other headgroups are also present to varying degrees and can include phosphatidylserine (PS) phosphatidylethanolamine (PE) and phosphatidylglycerol (PG). These alternate headgroups often confer specific biological functionality that is highly context-dependent. For instance, PS presence on the extracellular membrane face of erythrocytes is a marker of cell apoptosis, whereas PS in growth plate vesicles is necessary for the nucleation of hydroxyapatite crystals and subsequent bone mineralization. Unlike PC, some of the other headgroups carry a net charge, which can alter the electrostatic interactions of small molecules with the bilayer.

The primary role of the lipid bilayer in biology is to separate aqueous compartments from their surroundings. The partitioning ability of the lipid bilayer is based on the fact that hydrophilic molecules cannot easily cross the hydrophobic bilayer core. Because of natural membrane differences between prokaryotes and eukaryotes, and between species within the kingdoms, it is possible to selectively target vesicles or liposomes based on their membrane composition.

Most polar molecules have low solubility in the hydrocarbon core of a lipid bilayer and, as a consequence, have low permeability coefficients across the bilayer. This effect is particularly pronounced for charged species, which have even lower permeability coefficients than neutral polar molecules. Anions typically have a higher rate of diffusion through bilayers than cations. Compared to ions, water molecules actually have a relatively large permeability through the bilayer, as evidenced by osmotic swelling. When a cell or vesicle with a high interior salt concentration is placed in a solution with a low salt concentration it will swell and eventually burst. Such a result would not be observed unless water was able to pass through the bilayer with relative ease. Small uncharged apolar molecules diffuse through lipid bilayers many orders of magnitude faster than ions or water. This applies both to fats and organic solvents like chloroform and ether. Regardless of their polar character larger molecules diffuse more slowly across lipid bilayers than small molecules.

Some molecules or particles are too large or too hydrophilic to pass through a lipid bilayer. Other molecules could pass through the bilayer but must be transported rapidly in such large numbers that channel-type transport is impractical. In both cases, these types of cargo can be moved across the cell membrane through fusion or budding of vesicles. When a vesicle is produced inside the cell and fuses with the plasma membrane to release its contents into the extracellular space, this process is known as exocytosis. In the reverse process, a region of the cell membrane will dimple inwards and eventually pinch off, enclosing a portion of the extracellular fluid to transport it into the cell. Endocytosis and exocytosis rely on very different molecular machinery to function, but the two processes are intimately linked and could not work without each other. The primary mechanism of this interdependence is the large amount of lipid material involved. In a typical cell, an area of bilayer equivalent to the entire plasma membrane will travel through the endocytosis/exocytosis cycle in about half an hour.

Lipid bilayers are large enough structures to have some of the mechanical properties of liquids or solids. The area compression modulus Ka, bending modulus Kb, and edge energy Lambda, can be used to describe them. Solid lipid bilayers also have a shear modulus, but like any liquid, the shear modulus is zero for fluid bilayers. These mechanical properties affect how the membrane functions. Ka and Kb affect the ability of proteins and small molecules to insert into the bilayer, and bilayer mechanical properties have been shown to alter the function of mechanically activated ion channels. Bilayer mechanical properties also govern what types of stress a cell can withstand without tearing. Although lipid bilayers can easily bend, most cannot stretch more than a few percent before rupturing.

The hydrophobic attraction of lipid tails in water is the primary force holding lipid bilayers together. Thus, the elastic modulus of the bilayer is primarily determined by how much extra area is exposed to water when the lipid molecules are stretched apart. Ka varies strongly with osmotic pressure but only weakly with tail length and unsaturation. In contrast to Ka, which is a measure of how much energy is needed to stretch the bilayer, Kb is a measure of how much energy is needed to bend or flex the bilayer. Formally, bending modulus is defined as the energy required to deform a membrane from its intrinsic curvature to some other curvature. Intrinsic curvature is defined by the ratio of the diameter of the head group to that of the tail group. For two-tailed PC lipids, this ratio is nearly one so the intrinsic curvature is nearly zero. If a particular lipid has too large a deviation from zero intrinsic curvature it will not form a bilayer and will instead form other phases such as micelles or inverted micelles. Addition of small hydrophilic molecules like sucrose into mixed lipid lamellar liposomes made from galactolipid-rich thylakoid membranes destabilizes bilayers into micellar phase. Lambda is a measure of how much energy it takes to expose a bilayer edge to water by tearing the bilayer or creating a hole in it. The origin of this energy is the fact that creating such an interface exposes some of the lipid tails to water, but the exact orientation of these border lipids is unknown. There is some evidence that both hydrophobic (tails straight) and hydrophilic (heads curved around) pores can coexist.

Fusion is the process by which two lipid bilayers merge, resulting in one connected structure. If this fusion proceeds completely through both leaflets of both bilayers, a water-filled bridge is formed, and the solutions contained by the bilayers can mix. Alternatively, if only one leaflet from each bilayer is involved in the fusion process, the bilayers are said to be hemifused.

In studies of molecular and cellular biology it is often desirable to artificially induce fusion. The addition of polyethylene glycol (PEG) causes fusion without significant aggregation or biochemical disruption.

To date, the most successful commercial application of lipid bilayers has been the use of liposomes for drug delivery, especially for cancer treatment. (Note-the term “liposome” is in essence synonymous with “vesicle” except that vesicle is a general term for the structure whereas liposome refers to only artificial not natural vesicles) The basic idea of liposomal drug delivery is that the drug is encapsulated in solution inside the liposome then the patient is exposed. These drug-loaded liposomes may bind at the target site and rupture, releasing the drug.

The first generation of drug delivery liposomes had a simple lipid composition and suffered from several limitations. Refinement of the lipid composition to tune fluidity, surface charge density, and surface hydration resulted in vesicles that adsorb fewer proteins from serum and thus are less readily recognized by the immune system. The most significant advance in this area was the grafting of polyethylene glycol (PEG) onto the liposome surface to produce “stealth” vesicles, which circulate over long times without immune or renal clearing.

The terms “liposome,” “liposomal” and related terms as used herein are characterized by an interior aqueous space sequestered from an outer medium by one or more bilayer membranes forming a vesicle. en.wikipedia.org/wiki/Liposome. In certain embodiments, the interior aqueous space of the liposome is substantially free of a neutral lipid, such as triglyceride, non-aqueous phase (oil phase), water-oil emulsions, a second liposome or other mixtures containing non-aqueous phase. Non-limiting examples of liposomes include small unilamellar vesicles (SUV), large unilamellar vesicles (LUV), and multi-lamellar vesicles (MLU) with an average diameter ranges from 50-500 nm, 50-450 nm, 50-400 nm, 50-350 nm, 50-300 nm, 50-250 nm, 50-200 nm, 100-500 nm, 100-450 nm, 100-400 nm, 100-350 nm, 100-300 nm, 100-250 nm or 100-200 nm, all of which are capable of passing through sterile filters.

A unilamellar liposome is a spherical liposome, a vesicle, bounded by a single bilayer of an amphiphilic lipid or a mixture of such lipids, containing aqueous solution inside the chamber. en.wikipedia.org/wiki/Unilamellar_liposome. Unilamellar liposomes are used to study biological systems and to mimic cell membranes, and are classified into three groups based on their size: small unilamellar liposomes/vesicles (SUVs) that with a size range of 20-100 nm, large unilamellar liposomes/vesicles (LUVs) with a size range of 100-1000 nm and giant unilamellar liposomes/vesicles (GUVs) with a size range of 1-200 μm. GUVs are mostly used as models for biological membranes in research work. Animal cells are 10-30 μm and plant cells are typically 10-100 μm. Even smaller cell organelles such as mitochondria are typically 1-2 μm. Therefore, a proper model should account for the size of the specimen being studied. In addition, the size of vesicles dictates their membrane curvature which is an important factor in studying fusion proteins. SUVs have a higher membrane curvature and vesicles with high membrane curvature can promote membrane fusion faster than vesicles with lower membrane curvature such as GUVs.

The composition and characteristics of the cell membrane varies in different cells (plant cells, mammalian cells, bacterial cells, etc.). In a membrane bilayer, often the composition of the phospholipids is different between the inner and outer leaflets. Phosphatidylcholine, phosphatidylethanolamine, phosphatidylscrine, phosphatidylinositol, and sphingomyelin are some of the most common lipids most animal cell membranes. These lipids are widely different in charge, length, and saturation state. The presence of unsaturated bonds (double bonds) in lipids for example, creates a kink in acyl chains which further changes the lipid packing and results in a looser packing. Each lipid bilayer structure is comparable to lamellar phase lipid organization in biological membranes, in general. In contrast, multilamellar liposomes (MLVs), consist of many concentric amphiphilic lipid bilayers analogous to onion layers, and MLVs may be of variable sizes up to several micrometers.

There are several methods to prepare unilamellar liposomes and the protocols differ based on the type of desired unilamellar vesicles. Different lipids can be bought either dissolved in chloroform or as lyophilized lipids. In the case of lyophilized lipids, they can be solubilized in chloroform. Lipids are then mixed with a desired molar ratio. Then chloroform is evaporated using a gentle stream of nitrogen (to avoid oxygen contact and oxidation of lipids) at room temperature. A rotary evaporator can be used to form a homogeneous layer of liposomes. This step removes the bulk of chloroform. To remove the residues of trapped chloroform, lipids are placed under vacuum from several hours to overnight. Next step is re-hydration where the dried lipids are re-suspended in the desired buffer. Lipids can be vortexed for several minutes to ensure that all the lipid residues get re-suspended. SUVs can be obtained in via two methods. Either by sonication (for instance with 1 second pulses in 3 Hz cycles at a power of 150 W) or by extrusion. In extrusion method, the lipid mixture is passed through a membrane for 10 or more times. Depending on the size of the membrane, either SUVs or LUVs can be obtained. Keeping vesicles under argon and away from oxygen and light can extend their lifetime.

Phospholipid liposomes are used as targeted drug delivery systems. Hydrophilic drugs can be carried as solution inside the SUVs or MLVs and hydrophobic drugs can be incorporated into lipid bilayer of these liposomes. If injected into circulation of human/animal body, MLVs are preferentially taken up phagocytic cells, and thus drugs can be targeted to these cells. For general or overall delivery, SUVs may be used. For topical applications on skin, specialized lipids like phospholipids and sphingolipids may be used to make drug-free liposomes as moisturizers, and with drugs such as for anti-ultraviolet radiation applications.

Bilayer membranes of liposomes are typically formed by at least one lipid, i.e., amphiphilic molecules of synthetic or natural origin that comprise spatially separated hydrophobic and hydrophilic domains. Examples of lipid, including but not limited to, dialiphatic chain lipids, such as phospholipids, diglycerides, dialiphatic glycolipids, single lipids such as sphingomyelin and glycosphingolipid, and combinations thereof. Examples of phospholipid according to the present disclosure include, but not limited to, 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1-palmitoyl-2-stearoyl-sn-glycero-3-phosphocholine (PSPC), 1-palmitoyl-2-oleoyl-sn-glycero-3-phosphatidylcholine (POPC), 1,2-distearoyl-sn-glycero-3-phosphocholine (DSPC), 1,2-dioleoyl-sn-glycero-3-phosphocholine (DOPC), hydrogenated soy phosphatidylcholine (HSPC), 1,2-dimyristoyl-sn-glycero-3-phospho-(1′-rac-glycerol) (sodium salt) (DMPG), 1,2-dipalmitoyl-sn-glycero-3-phospho-(1′-rac-glycerol) (sodium salt) (DPPG), 1-palmitoyl-2-stearoyl-sn-glycero-3-phospho-(1′-rac-glycerol) (sodium salt) (PSPG), 1,2-distearoyl-sn-glycero-3-phospho-(1′-rac-glycerol) (sodium salt) (DSPG), 1,2-dioleoyl-sn-glycero-3-phospho-(1′-rac-glycerol) (DOPG), 1,2-dimyristoyl-sn-glycero-3-phospho-L-serine (sodium salt) (DMPS), 1,2-dipalmitoyl-sn-glycero-3-phospho-L-serine (sodium salt) (DPPS), 2-distearoyl-sn-glycero-3-phospho-L-serine (sodium salt) (DSPS), 1,2-dioleoyl-sn-glycero-3-phospho-L-serine (DOPS), 1,2-dimyristoyl-sn-glycero-3-phosphate (sodium salt) (DMPA), 1,2-dipalmitoyl-sn-glycero-3-phosphate (sodium salt) (DPPA), 1,2-distearoyl-sn-glycero-3-phosphate (sodium salt) (DSPA), 1,2-dioleoyl-sn-glycero-3-phosphate (sodium salt) (DOPA), 1,2-dipalmitoyl-sn-glycero-3-phosphoethanolamine (DPPE), N-(carbonyl-methoxypolyethyleneglycol)-1,2-dipalmitoyl-sn-glycero-3-phosphoethanolamine (PEG-DPPE), 1-palmitoyl-2-oleoyl-sn-glycero-3-phosphoethanolamine (POPE), 1,2-distearoyl-sn-glycero-3-phosphoethanolamine (DSPE), N-(carbonyl-methoxypolyethyleneglycol)-1,2-distearoyl-sn-glycero-3-phosphoethanolamine (PEG-DSPE), 1,2-dioleoyl-sn-glycero-3-phosphoethanolamine (DOPE), 1,2-dipalmitoyl-sn-glycero-3-phospho-(1′-myo-inositol) (ammonium salt) (DPPI), 1,2-distearoyl-sn-glycero-3-phosphoinositol (ammonium salt) (DSPI), 1,2-dioleoyl-sn-glycero-3-phospho-(1′-myo-inositol) (ammonium salt) (DOPI), cardiolipin, L-α-phosphatidylcholine (EPC), and L-α-phosphatidylethanolamine (EPE).

In some embodiments, the lipid is a lipid mixture of one or more of the foregoing lipids, or mixtures of one or more of the foregoing lipids with one or more other lipids not listed above, membrane stabilizers or antioxidants.

The mole percent of the lipid in the bilayer membrane may be equal or less than about 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45 or any value or range of values therebetween (e.g., about 45-85%, about 45-80%, about 45-75%, about 45-70%, about 45-65%, about 50-85%, about 50-80%, about 50-75%, about 50-70%, or about 50-65%).

In some embodiments, the lipid of the bilayer membrane comprises a mixture of a first lipid and a second lipid. The first lipid is selected from the group consisting essentially of phosphatidylcholine (PC), HSPC, DSPC, DPPC, DMPC, PSPC and a combination thereof. The second lipid is selected from the group consisting essentially of a phosphatidylethanolamine, phosphatidylglycerol, PEG-DSPE, DPPG and a combination thereof. In other embodiments, the mole percent of the first lipid in the bilayer membrane is equal or less than about 84.9, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30 or any value or range of values therebetween (e.g., about 30-84.9%, about 30-80, about 35-75, about 40-70, about 30-70, about 30-60, about 35-84.9%, about 35-80, about 35-70, about 35-60, about 40-84.9%, about 40-80, about 40-75 or about 40-62) and the mole percent of the second lipid in the bilayer membrane is equal to or less than about 50, 40, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 to 0.1 or any value or range of values therebetween (e.g., about 0.1-30%, about 0.1-25%, about 0.1-23%, about 0.5-30%, about 0.5-25%, about 0.5-23%, about 0.7-30%, about 0.7-25% or about 0.7-23%).

Bilayer membranes of liposomes may further comprise less than about 55 mole percentage of steroids, preferably a bile acid, e.g., cholic acid or its salt, e.g., sodium cholate. In certain embodiments, the percentage of steroid in the bilayer membrane is about 5-55%, about 10-50%, about 15-40%, about 20-35, about 17-30%, about 17-25%, or about 20%, In one exemplary embodiment, the mole percentage of the lipid and cholic acid in the bilayer membrane is about 50-95%:5-50%, 60-90%:10-40%, 70-85%:15-30%, 80:20%.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali (e.g., sodium, potassium) or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like. The salt, e.g., of cholic acid is selected based on solubility, pKa (disassociation constant), toxicity, interaction with the pharmacological agent and other components, and other known criteria.

The gel-forming substance of the present invention can e.g., be selected from the group consisting of agar, alginates, alginic acids, Arabic gum, gelatine, starch, tragacanth gum, methylcelluloses, hydroxyethylcelluloses, carboxymethylcelluloses, polyacrylic acids and/or combinations thereof. In preferred embodiments, acrylic acid polymers are applied. Polymers complying the USP Carbomer 940 monograph like Carbopol 908NF or Carbomer 940) are preferred from this group. See, US 2014/0141063. The gel-forming substances may comprise polyacrylates, polymethacrylates, polyacrylic acids, polymethacrylic acids, polyvinylalcohols and combinations thereof. Polyacrylic acids are particularly preferred. In preferred embodiments of the invention, gel-forming substances are used in the form of hydrogels. A hydrogel, as used in the present invention, is a gel on the basis of a hydrophilic composition or compound, which is capable of absorbing and/or releasing a certain amount of liquid, in particular water. The pH may be in the range from 3 to 7, more preferably from 4 to 6.5 and even more preferably in the range from 5 to 6.

The gel-forming substance is present in the preparation according to the invention at between about 0.1% and about 10%, preferably between about 0.5% and about 5%, more preferably between about 1.0% and about 5%, and most preferably 3.5%. All these percentages are wt.-%, based on total preparation weight.

Lecithin and other phospholipids may be used to prepare liposomes comprising the compositions as described herein. In some instances, the liposomes comprise phospholipids. In some instances, the liposomes comprise phospholipids, fatty acids, or fatty acid groups. The phospholipids, fatty acids, or fatty acid groups may be saturated. The phospholipids, fatty acids, or fatty acid groups may be unsaturated. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals can be dissolved into the phospholipid bilayer membrane. The lipid bilayers of the liposomes may deliver the compositions as described herein.

Liposome gels are known in the art. For example, liposome gels comprising lidocaine hydrochloride, an anesthetic agent, have been produced. See Glavas-Dodov, M., et al., Bulletin of the Chemists and Technologists of Macedonia, (2005), 24, 59-65.

The liposomes are formed from one or more naturally occurring or synthetic lipid compounds, or a mixture thereof. Suitable lipids include detergents, surfactants, soaps, phospholipids, ether lipids, glycoglycerolipids, etc. More specific examples of suitable lipids include unsaturated fatty acids (e.g., myristoleic, palmitoleic, elaidic, petroselinic, oleic, vaccenic, gondoic, erucic, nervonic, linoleic, gamma-linolenic, linolenic, arachidonic, eicosapentaenoic, docosahexaenoic acids, etc.), the corresponding fatty acid derivatives (e.g., amides, esters, etc.), the corresponding sulfonic acids, the corresponding sulfonic acid derivatives (e.g., sulfonamides, sulfonate esters, etc.), the corresponding fatty alcohols, etc.

In some embodiments the lipid includes one or more unsaturated acyl moieties. In other embodiments the lipid is a phospholipid, such as natural or synthetic phospholipids, saturated or unsaturated phospholipids, or phospholipid-like molecules. The phospholipid typically includes one or more saturated or unsaturated acyl moieties. In some embodiments the unsaturated acyl moiety is C₁₂-C₂₄ alkenyl. In some embodiments the saturated acyl moiety is C₁₂-C₂₄ alkyl. Particularly suitable phospholipids include e.g., soybean lecithin, egg lecithin, lecithin, lysolecithin, phosphatidylserine, phosphatidylethanolamine, phosphatidylcholine and phosphatidylinositol, phosphatidylglycerol, phosphatidylacid, etc. In some embodiments, the phospholipids are mixed with a sterol such as cholesterol, which can stabilize the phospholipid system. In some embodiments, the lipid is chemically or physically modified. Modifications alter the properties of the lipid and of the resulting liposome vesicles. Methods of modifying lipids are known in the art of liposomal formulations. In preferred embodiments, the gel composition includes a phospholipid such as those available under the trade names Phospholipon® 90G, Phospholipon® 19H, NanoSolve® or Lipoid SPC®. In some embodiments the phosphoipids are mixed with alcohol. Ethosomes are composed mainly of phospholipids, (phosphatidylcholine, phosphatidylserine, phosphatitidic acid), high concentration of ethanol and water. The high concentration of ethanol makes the ethosomes unique, as ethanol is known for its disturbance of skin lipid bilayer organization; therefore, when integrated into a vesicle membrane, it gives that vesicle the ability to penetrate the stratum corneum. Additional non-phosphorous-containing lipids suitable for use in the compositions of the present invention include stearylamines, fatty acids, fatty acid amides, fatty alcohol ethers, fatty alcohols, fatty alcohol phosphates etc. Suitable non-phosphorous-containing lipids usable as the surfactant include but are not limited to C₆-C₂₂ fatty acids and alcohols, such as stearyl alcohol, capric acid, caprylic acid, lauric acid, myristic acid, stearic acid, oleic acid, linoleic acid, linolenic acid, arachnidoic acid, behenic acid, and their corresponding pharmaceutically acceptable salts. In some embodiments the non-phosphorous-containing lipids include surfactants such as sodium dioctyl sulfosuccinate, sodium lauryl sulfate, amide esters, (e.g., lauric acid diethanolamide, sodium lauryl sarcosinate, lauroyl carnitine, palmitoyl carnitine and myristoyl carnitine), esters with hydroxy-acids (e.g., sodium stearoyl lactylate), sugar esters (e.g., lauryl lactate, glucose monocaprylate, diglucose monocaprylate, sucrose laurate, sorbitan monolaurate, sorbitan monopalmitate, sorbitan monooleate sorbitansesquioleate, sorbitan monostearate and sorbitan tristearate), lower alcohol fatty acid esters (e.g., ethyl oleate, isopropyl myristate and isopropyl palmitate) esters with propylene glycol (e.g., propylene glycol monolaurate, propylene glycol ricinoleate, propylene glycol monooleate, propylene glycol monocaprylate, propylene glycol dicaprylate/dicaprate and propylene glycol dioctanoate), esters with glycerol (e.g., glyceryl monooleate, glyceryl ricinoleate, glyceryl laurate, glyceryl dilaurate, glyceryl dioleate, glycerol monolinoleate, glyceryl mono/dioleate, glyceryl caprylate/caprate, caprylic acid mono/diglycerides, mono-and diacetylated monoglycerides), triglycerides (e.g., corn oil, almond oil, soybean oil, coconut oil, castor oil, hydrogenated castor oil, hydrogenated coconut oil, Pureco 100, Hydrokote AP5, Captex 300, 350, Miglyol 812, Miglyol 818 and Gelucire 33/01), mixtures of propylene glycol esters and glycerol esters (e.g., mixture of oleic acid esters of propylene glycol and glycerol and polyglycerized fatty acids such as polyglyceryl oleate), polyglyceryl-2 dioleate, polyglyceryl-10 trioleate, polyglyceryl-10 laurate, polyglyceryl-10 oleate, and polyglyceryl-10 mono, dioleate (Caprol® PEG 860)). Other suitable non-phosphorous-containing lipids include polyethoxylated fatty acids, (e.g., PEG-8 laurate, PEG-8 oleate, PEG-8 stearate, PEG-9 oleate, PEG-10 laurate, PEG-5 oleate, PEG-10 oleate, PEG-12 laurate, PEG-12 oleate, PEG-15 oleate, PEG-20 laurate and PEG-20 oleate) PEG-fatty acid diesters (e.g., PEG-20 dilaurate, PEG-20 dioleate, PEG-20 distearate, PEG-32 dilaurate and PEG-32 dioleate) PEG-fatty acid mono- and di-ester mixtures, polyethylene glycol glycerol fatty acid esters (e.g., PEGylated glycerol 12-acyloxy-stearate, PEG-20 glyceryl laurate, PEG-30 glyceryl laurate, PEG-40 glyceryl laurate, PEG-20 glyceryl oleate and PEG-30 glyceryl oleate) and alcohol-oil transesterification products (e.g., polyoxyl 40 castor oil, polyoxyl 35 castor oil, PEG-25 trioleate, PEG-60 corn glycerides, PEG-60 almond oil, PEG-40 palm kernel oil, PEG-50 castor oil, PEG-50 hydrogenated castor oil, PEG-60 hydrogenated castor oil, PEG-8 caprylic/capric glycerides, lauroyl macrogol-32 glycerides, linoleoyl macrogolglycerides), stearoyl macrogol-32 glycerides, and PEG-6 caprylic/capric glycerides). The lipid may be Imwitor® 375 (glyceryl citrate/lactate/linoleate/oleate), Polyglyceryl-3 Polyricinoleate or ethyl oleate.

In certain embodiments of the invention the composition includes a hydrophilic non-ionic surfactant combined with a lipid or a lipophilic non-ionic surfactant. Participation of non-ionic surfactants instead of phospholipids in the lipid bilayer results in niosomes. The term “hydrophilic surfactant” means an oil-in-water surfactant with a hydrophilic-lipophilic balance (HLB) value of 9-18, and “lipophilic surfactant” means a water-in-oil surfactant with an HLB value of 1.5-9. By way of an example, polysorbate 80 has an HLB value of 15 and is therefore a hydrophilic surfactant, whereas sorbitan trioleate has an HLB value of 1.8 and is therefore a lipophilic surfactant. The HLB of mixed surfactants is calculated according to their relative weightings (by volume) e.g., a 1:1 mixture by volume of polysorbate 80 and sorbitan trioleate has a HLB of 8.4.

The composition may include a hydrophilic non-ionic surfactant in an amount of from about 1% to about 40% by weight of the composition, optionally from about 2% to about 15% by weight of the composition. Preferably, the composition includes a hydrophilic non-ionic surfactant in an amount of from about 2% to about 10% by weight of the composition, such as from about 2.5% to about 5% by weight of the composition. The hydrophilic non-ionic surfactant may be a polyethylene glycol ester of a vegetable oil containing at least 20 moles of ethylene oxide groups/mole of glyceride. Suitable polyethylene glycol esters are typically selected from polyoxyethylene castor oil derivatives (e.g., PEG 20, 30, 35, 38, 40, 50 and 60 castor oil or PEG 20, 25, 30, 40, 45, 50, 60 and 80 hydrogenated castor oil), PEG 20 and 60 corn glycerides, PEG 20 and 60 almond glycerides, PEG 40 palm kernel oil, sodium laurate sulfate, sucrose esters (e.g., sucrose stearate, sucrose distearate, sucrose cocoate or sucrose monolaurate), PEG cocoglyceride, PEG 8 caprylocaprate, polyglyceryl esters and linolenamide DEA. In a preferred embodiment, the hydrophilic non-ionic surfactant is sucrose distearate, such as that available under the trade name Sisterna® SP30.

In certain embodiments, the hydrophilic non-ionic surfactant may be a mixture of acrylamide acryloyldimethyl taurate copolymer, isohexadecane and polysorbate 80, such as that available under the trade name SEPINEO™ P600. The hydrophilic non-ionic surfactant may be an alkylpolyglucoside, such as that available under the trade name SEPINEO™ SE68.

A lipophilic non-ionic surfactant may be present in an amount of from about 0.1% to about 5% by weight of the composition. Surfactants are generally irritants, and so it is preferred to use only low levels of certain surfactants. However, some lipophilic non-ionic surfactants, such as monoglyceride esters, are less irritative and so can be present in higher amounts without causing significant levels of skin irritation.

The lipophilic non-ionic surfactant may be selected from monoglyceride esters of C₆-C₂₂ fatty acids (e.g., glyceryl monocaprylate, glyceryl monocaprate, glyceryl monostearate, glyceryl monobehenate), diglyceride esters of C₆-C₂₂ fatty acids (e.g., glyceryl dilaurate), mono- and diglyceride esters of C₆-C₂₂ fatty acids (e.g., caprylic/capric mono- and diglyceride, glyceryl mono- and diricinoleate), propylene glycol esters of C₆-C₂₂ fatty esters (e.g., propylene glycol monocaprylate, propylene glycol monolaurate), dialkylene glycol monoalkyl ethers (e.g., diethylene glycol monoethyl ether), polyglyceryl C₆-C₂₂ fatty acid esters (e.g., polyglyceryl-3-diisostearate), polyethylene glycol esters of a triglyceride/vegetable oil containing 4 to 8 moles of ethylene oxide groups/mole of glyceride (e.g., PEG-6 corn oil, PEG-6 almond oil, PEG-6 apricot kernel oil, PEG-6 olive oil, PEG-6 peanut oil, PEG-6 palm kernel oil, hydrogenated palm kernel oil, PEG-6 triolein, PEG-8 corn oil), polysorbates (e.g., polysorbate 20, polysorbate 40, polysorbate 60, polysorbate 80). In a preferred embodiment, the lipophilic non-ionic surfactant is a sorbitan ester, such as that available under the trade name Span® 120. In a preferred embodiment, the lipophilic non-ionic surfactant is an oleoyl macrogol-6 glyceride, such as that available under the trade name Labrafil® M1944 or a lauroyl polyoxyl-6 glyceride, such as that available under the trade name Labrafil® M2130.

The compositions of the invention may include a mixture of different lipids, e.g., one or more phospholipids and one or more non-phosphorous-containing lipids within the same composition.

The aqueous composition component may include an aqueous buffer solution. The use of buffer solutions means that fluctuations in pH can be minimized and thus the pH can be kept more readily within the desired pH range, such as at a pH of between pH 6 and pH 8.2.

The composition may include one or more emulsifiers selected from e.g., polyacrylates, polycarbophils, poloxamers, hyaluronic acid, xanthan, natural polysaccharides, chitosan and cellulose-derivatives. Suitable cellulose-derivative viscosity enhancers include hydroxyalkyl cellulose polymers (e.g., hydroxymethyl cellulose, hydroxyethyl cellulose, hydroxypropyl cellulose (hypromellose) and hydroxypropylmethyl cellulose), carboxymethyl cellulose, methylhydroxyethyl cellulose and methylcellulose, carbomer (e.g., Carbopol®), and carrageenans. In certain preferred embodiments, the emulsifier is hydroxyethyl cellulose, such as that available under the trade name Natrosol® (e.g., Natrosol® 250 HX, Natrosol® PLUS CS, Grade 300 etc.) and Methocel®. In certain preferred embodiments, the emulsifier is hydroxypropyl cellulose, such as that available under the trade name Klucel® and Methocel®. Typically, the emulsifier is present in an amount of from about 1% to about 20% by weight of the composition, such as about 1%, 3%, 5%, 10%, 15% or 20% by weight of the composition. The composition may include more than one viscosity enhancers, such as two or three viscosity enhancers.

It may be useful to include one or more penetration enhancers. Typical penetration enhancers include propylene carbonate, transcutol, pyrrolidones such as N-methylpyrrolidone or N-hydroxyalkylpyrrolidone, azone, menthol, eucalyptol, nicotinamide, glycerol, mono-di- or polyglycols, ethylacetate or Eugenol. A particularly preferred penetration enhancer is α-tocopherol. A penetration enhancer may be present in an amount of from about 0.01% to about 20% by weight of the composition, such as from about 0.1% to about 15%, e.g., about 0.1%, about 0.5%, about 1%, about 1.5%, about 2%, about 2.5%, about 3%, or about 5% by weight of the composition.

The phospholipids used to prepare the liposomal compositions described herein may comprise a transition phase temperature of about 10° C. to about 25° C. In some instances, the phospholipids comprise a transition phase temperature of about 10° C., 12° C., 14° C., 16° C., 18° C., 20° C., 22° C., 24° C., 26° C., 28° C., 30° C., 32° C., 34° C., 36° C., 38° C., 40° C., or more than 40° C. In some instances, the phospholipids comprise a transition phase temperature in a range of about 10° C. to about 40° C., about 12° C. to about 36° C., about 14° C. to about 32° C., about 16° C. to about 20° C., or about 21° C. to about 25° C.

Methods for generation of liposomal compositions as described herein may result in an entrapment efficacy of no more than 100%. In some instances, the entrapment efficacy is no more than 50%, 60%, 70%, 80%, 90%, 95%, 99%, or 99.5%.

Described herein are liposomal compositions, wherein the active agent comprises a percentage of the composition. In some embodiments, the active agent is provided at least or about 0.0001%, 0.0005%, 0.00055%, 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% of the composition. In some embodiments, the active agent is provided at least or about 10%, 11%, 12%, 13%, 14%, 15%, 16%, 17%, 18%, 19%, 20%, 22%, 24%, 26%, 28%, 30% or more than 30% of the composition. In some embodiments, the active agent is provided in a range of about 0.1% to about 2.5%, about 0.2% to about 1%, about 0.3% to about 0.8%, or about 0.5% by weight of the aqueous phase surrounded by the membrane.

Described herein are liposomal compositions, wherein the liposomes comprise a percentage of the composition. In some embodiments, the liposomes are provided at least or about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or more than 30% of the composition. In some embodiments, the liposomes are provided in a range of about 5% to about 90%, about 10% to about 80%, about 20% to about 70%, about 30% to about 60%, about 10% to about 30%, or about 20% to about 40%. In some embodiments, the liposomes are provided at about 30%.

Liposomal compositions as described herein, in some embodiments, comprise an average particle size of at most 220 nanometers (nm). In some instances, the average particle size is at most 50 nm, 55 nm, 60 nm, 65 nm, 70 nm, 75 nm, 80 nm, 86 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 230 nm, 240 nm, 250 nm, 260 nm, 270 nm, 280 nm, 290 nm, 300 nm, 320 nm, 340 nm, 360 nm, 380 nm, or 400 nm. In some instances, the average particle size is about 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 230 nm, 240 nm, 250 nm, 260 nm, 270 nm, 280 nm, 290 nm, 300 nm, 320 nm, 340 nm, 360 nm, 380 nm, or 400 nm. In some instances, the average particle size is in a range of about 50 nm to about 500 nm, about 100 nm to about 400 nm, about 150 nm to about 220 nm, about 180 nm to about 220 nm, or about 190 nm to about 210 nm.

As used herein, the term “local anesthetic” means a drug which provides local sensory nerve blockage. Local anesthetics cause reversible blockage of conduction and/or initiation of action potentials typically by actions related to the interference with voltage gated sodium channels. Lipid solubility appears to be the primary determinant of intrinsic anesthetic potency. Chemical compounds which are highly lipophilic tend to penetrate the nerve membrane more easily, such that fewer molecules are required for conduction blockade resulting in enhanced potency.

Chemically most local anesthetics are esters or amides. Esters include, but are not limited to, procaine, tetracaine, and chloroprocaine. They are hydrolyzed in plasma by pseudo-cholinesterase. Amides include, but are not limited to, lidocaine, mepivicaine, prilocaine, bupivacaine, and etidocaine. These compounds are often referred to as the “caine alkaloids”. Caine alkaloids generally have high first pass metabolisms. The liver rapidly metabolizes the drug and the kidneys excrete the metabolites and/or unchanged drug.

A number of different local anesthetics can be used, including lidocaine, pramoxine, prilocaine, benzocaine, tetracaine, bupivanor, betacaine, topicaine, procaine, chloroprocaine, cocaine, dibucaine, mepivacaine, bupivacaine, levobupivacaine, ropivacaine, articaine, etidocaine, butamben, oxybuprocaine, proxymetacaine (proparacaine), and xylocaine. In the preferred embodiment, the anesthetic is lidocaine, most preferably in the form of the free base, although it may be possible to use a salt, for example, the hydrochloride, hydrobromide, acetate, citrate, or sulfate salt.

A polydispersity index (PdI) of a liposomal composition as described herein, in some embodiments, is in a range of 0 to about 0.2. In some instances, the polydispersity index is about 0.01, 0.025, 0.05, 0.1, 0.25, 0.3, 0.35, 0.4, 0.45, 0.5, 0.55, 0.6, 0.65, 0.7, 0.75, or 0.8. In some instances, the polydispersity index is in a range of about 0.01 to about 0.8, about 0.025 to about 0.75, about 0.05 to about 0.6, or about 0.1 to about 0.3.

In some instances, an intercept of a liposomal composition as described herein is in a range of about 0.85 to about 0.95. In some instances, the intercept is the amplitude. In some instances, the intercept is at least or about 0.65, 0.70, 0.75, 0.80, 0.85, 0.90, or 0.95.

Phosphatidylcholine (herein abbreviated “PC”) is a basic component of cell membrane bilayers and the main phospholipid circulating in the plasma of blood. Phosphatidylcholine typically has a phospholipid structure with a choline head group and a glycerophosphoric acid tail group. The tail group can be saturated or unsaturated. More than one tail group may be present in the phosphatidylcholine in some cases, and the tail groups may be the same or different. Specific non-limiting examples of phosphatidylcholines that could be used include one or a mixture of stearic, palmitic, margaric, and/or oleic acid diglycerides linked to a choline ester head group.

Phosphatidylcholines are a member of a class of compounds called lecithins. Typically, a lecithin is a composed of phosphoric acid, choline, fatty acids, glycerol, glycolipids, triglycerides, and/or phospholipids. In some cases, other lecithins may be used, in addition to or instead of a phosphatidylcholine. Non-limiting examples of other lecithins include phosphatidylethanolamine, phosphatidylinositol, or phosphatidic acid. Many commercial lecithin products are available, such as, for example, Lecithol®, Vitellin®, Kelecin®, and Granulestin®.

Some compositions may contain polyenylphosphatidylcholine (herein abbreviated “PPC”). In some cases, PPC can be used to enhance epidermal penetration. The term “polyenylphosphatidylcholine,” as used herein, means any phosphatidylcholine bearing two fatty acid moieties, wherein at least one of the two fatty acids is an unsaturated fatty acid with at least two double bonds in its structure, such as linoleic acid.

Certain types of soybean lecithin and soybean fractions, for example, can contain higher levels of polyenylphosphatidylcholine, with dilinoleoylphosphatidylcholine (18:2-18:2 phosphatidylcholine) as the most abundant phosphatidylcholine species therein, than conventional food grade lecithin. Such lecithins may be useful in formulating certain delivery compositions. In some embodiments, conventional soybean lecithin may be enriched with polyenylphosphatidylcholine, for instance, by adding soybean extracts containing high levels of polyenylphosphatidylcholine. As used herein, this type of phosphatidylcholine is called “polyenylphosphatidylcholine-enriched” phosphatidylcholine (hereinafter referred to as PPC-enriched phosphatidylcholine), even where the term encompasses lecithin obtained from natural sources exhibiting polyenylphosphatidylcholine levels higher than ordinary soybean varieties. These products are commercially available, for example, from American Lecithin Company, Rhone-Poulenc and other lecithin vendors. American Lecithin Company markets its products with a “U” designation, indicating high levels of unsaturation; Rhone-Poulenc's product is a soybean extract containing about 42% dilinoleoylphosphatidylcholine and about 24% palmitoyllinoleylphosphatidylcholine (16:0 to 18:2 of PC) as the major phosphatidylcholine components. Another example of a suitable polyenylphosphatidylcholine is NAT 8729 (also commercially available from vendors such as Rhone-Poulenc and American Lecithin Company).

Other components may also be present within the composition, in accordance with certain embodiments of the invention. For example, the composition may include volatile organic fluids, fatty acids, volatile aromatic cyclic compounds, high molecular weight hydrocarbons, or the like.

Any suitable amount of polyenylphosphatidylcholine or lecithin may be present within the composition. For example, at least about 0.25 wt %, at least about 0.5 wt %, at least about 1 wt %, at least about 2 wt %, at least about 3 wt %, at least about 5 wt %, at least about 8 wt %, at least about 10 wt %, at least about 20 wt %, at least about 30 wt %, at least about 40 wt %, at least about 50 wt %, at least about 60 wt %, at least about 70 wt %, at least about 80 wt %, or at least about 90 wt % of the composition can be polyenylphosphatidylcholine or lecithin. In some cases, the polyenylphosphatidylcholine or lecithin may be present at a concentration of no more than about 95 wt %, no more than about 90 wt %, no more than about 80 wt %, no more than about 70 wt %, no more than about 65 wt %, no more than about 60 wt %, no more than about 50 wt %, no more than about 40 wt %, no more than about 30 wt %, no more than about 20 wt %, or no more than about 10%. Combinations of any of these are also possible. For instance, the polyenylphosphatidylcholine or lecithin may be present at between about 8 wt % and about 65 wt %. In some embodiments, at least about 20 wt %, at least about 30 wt %, at least about 40 wt %, at least about 50 wt %, at least about 60 wt %, at least about 70 wt %, at least about 80 wt %, at least about 90 wt %, or about 100 wt % of all of the phosphatidylcholine or lecithin in the composition is polyenylphosphatidylcholine. PPC-enriched phosphatidylcholine may contribute to the stability of the composition, and/or enhance its penetration into the skin or other area, e.g., a mucosal surface.

In some embodiments, the liposome membrane comprises sodium cholate and soybean phosphatidylcholine (SPC). In some embodiments, the sodium cholate is provided at least or about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, or more than 40% by weight (wt.) In some embodiments, the sodium cholate is provided in a range of about 10% to about 30%, about 15% to about 25%, about 20%. In some embodiments, the phosphatidylcholine is provided in a range of 50% to 95%, about 55% to 90%, about 60% to 85%, about 65% to 80%, about 70% to 80%, and about 75% to 80%.

As used herein, the word “about” means a variation limit of −25%/+35% from the stated value. The word “substantially” means ±10%.

Liposomal compositions and methods as described herein, in some embodiments, are topical compositions. In some instances, the liposomal compositions are preservative free. In some embodiments, the liposomal formulation is an aqueous gel formulation. In some instances, the liposomal composition comprises a pH in a range of about 5 to about 8. In some instances, the liposomal composition comprises a pH of at least or about 2, 3, 4, 5, 6, 7, 8, 9, or 10.

The composition may also include one or more transdermal penetration enhancers. Examples of transdermal penetration enhancers include, but are not limited to, 1,3-dimethyl-2-imidazolidinone or 1,2-propanediol. Other examples include cationic, anionic, or nonionic surfactants (e.g., sodium dodecyl sulfate, polyoxamers, etc.); fatty acids and alcohols (e.g., ethanol, oleic acid, lauric acid, liposomes, etc.); anticholinergic agents (e.g., benzilonium bromide, oxyphenonium bromide); alkanones (e.g., n-heptane); amides (e.g., urea, N,N-dimethyl-m-toluamide); organic acids (e.g., citric acid); sulfoxides (e.g., dimethylsulfoxide); terpenes (e.g., cyclohexene); ureas; sugars; carbohydrates or other agents. The transdermal penetration enhancers can be present in any suitable amount within the composition. For example, at least about 10 wt %, at least about 20 wt %, at least about 30 wt %, at least about 40 wt %, or at least about 50 wt % of the composition may comprise one or more transdermal penetration enhancers. In some cases, no more than about 60 wt %, no more than about 50 wt %, no more than about 40 wt %, no more than about 30 wt %, no more than about 20 wt %, no more than about 10 wt %, no more than about 9 wt %, or no more than about 5 wt % of the composition comprises transdermal penetration enhancers. Combinations of any of these are also possible. For example, the composition may have between about 0 wt % and about 5 wt % of one or more transdermal penetration enhancers.

Fatty acids and alcohols can be employed to enhance penetration of the peptides, and to provide a silky feel to formulations, e.g., methanoic acid, ethanoic acid, propanoic acid, butanoic acid, isobutyric acid, pentanoic acid, hexanoic acid, heptanoic acid, octanoic acid, nonanoic acid, decanoic acid, myristoleic acid, isovaleric acid, palmitoleic acid, sapienic acid, oleic acid, elaidic acid, vaccenic acid, linoleic acid, linoelaidic acid, .alpha.-linolenic acid, arachidonic acid, eicosapentaenoic acid, erucic acid, docosahexaenoic acid, caprylic acid, capric acid, lauric acid, palmitic acid, stearic acid, arachidic acid, behenic acid, lignoceric acid, cerotic acid, medium chain fatty acids, e.g., C₆-C₁₂ fatty acids, or the like. Typical amounts when employed in liposomal compositions are from 1% by weight to 4% by weight.

Additionally, in certain embodiments, other antioxidants may be present as well within the composition, i.e., in addition to any one or more of the above. Non-limiting examples of antioxidants include ascorbic acid, resveratrol, lipoic acid, carotenoids, pyruvic acid and/or a pyruvate salt, glutathione and/or a glutathione salt, for example, sodium glutathione, lithium glutathione, magnesium glutathione, calcium glutathione, potassium glutathione, ammonium glutathione, and the like. In another set of embodiments, a composition may be applied to the skin that comprises lipoic acid and/or a salt thereof, for example, a sodium, lithium, magnesium, calcium, potassium, ammonium, etc. salt of lipoic acid. Still other non-limiting examples include various antioxidants such as oxalic acid, phytic acid, tannins, ascorbic acid, uric acid, caratones, alpha-tocopherol, ubiquinol, and the salts of any of these. In addition, it should be understood that in some cases, a composition may comprise more than one of the antioxidants discussed herein.

Other components can include anti-inflammatory agents, antioxidants, and solubility enhancers. Exemplary anti-irritation agents include, but are not limited to, panthenyl triacetate and naringenin. Panthenyl triacetate and naringenin are natural plant extracts that reduce redness and water loss through the skin. Typical amounts for anti-irritation agents when employed in liposomal compositions are from 1% by weight to 4% by weight.

Exemplary anti-inflammatory agents include, but are not limited to, Arnica montana extract. Arnica montana extract includes components such as essential oils, fatty acids, thymol, pseudoguaianolide sesquiterpene lactones and flavanone glycosides. It can exhibit an anti-inflammatory effect. Typical amounts for anti-inflammatory agents when employed in liposomal compositions are from 1% by weight to 4% by weight. Typical amounts for anti-inflammatory agents when employed in liposomal compositions are from 0.1% by weight to 2% by weight.

In some embodiments, the active ingredient(s) can be in admixture with a suitable carrier, diluent, or excipient, and can contain auxiliary substances such as wetting or emulsifying agents, pH buffering agents, gelling or viscosity enhancing additives, preservatives, scenting agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulations include, without limitation, monoglycerides, diglycerides, sulfatides, lysolecithin, phospholipids, saponin, bile acids, and the like. The presence of such additional components can influence the physical state, solubility, stability, rate of release, rate of clearance, and penetration of active ingredients.

The pharmaceutical excipients used in the topical preparations of the compositions may be selected from the group consisting of solvents, emollients and/or emulsifiers, oil bases, preservatives, antioxidants, tonicity adjusters, penetration enhancers and solubilizers, chelating agents, buffering agents, surfactants, one or more polymers, and combinations thereof. An exemplary chelating agent is disodium EDTA. In some embodiments, the disodium EDTA is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.)

Suitable solvents for an aqueous or hydrophilic liposomal composition include water; ethyl alcohol; isopropyl alcohol; mixtures of water and ethyl and/or isopropyl alcohols; glycerin; ethylene, propylene or butylene glycols; DMSO; and mixtures thereof. In some embodiments, glycerin is provided at least or about 1%, 2%, 3%, 4%, 5%, 6%, 7%, 8%, 9%, 10%, 11%, 12%, or more than 12%. In some embodiments, glycerin is provided at least or about 7%. In some embodiments, glycerin is provided in a range of about 1% to about 12%, about 2% to about 11%, or about 3% to about 10%. Suitable solvents for hydrophobic liposomal compositions include mineral oils, vegetable oils, and silicone oils. If desired, the compositions as described herein may be dissolved or dispersed in a hydrophobic oil phase, and the oil phase may then be emulsified in an aqueous phase comprising water, alone or in combination with lower alcohols, glycerin, and/or glycols. It is generally preferred to employ anhydrous compositions, as the presence of water can result in stinging upon administration to skin tissues subject to laser treatment, chemical peel, dermabrasion, or the like. Anhydrous formulations may also act to prevent the development of water-based irritant contact dermatitis in damaged or sensitive skin, which may produce rashes and skin irritation that may retard wound healing and improvement in skin quality. Tsai, T. F., Maibach, H. I. How irritant is water? An overview. Contact Dermatitis 41(6) (1999): 311-314 (describing contact dermatitis caused by water as an irritant). However, in certain embodiments it may be acceptable to provide water-based compositions, or to permit a limited amount of water to be present. For example, water may be present, but at amounts below the threshold at which a stinging sensation when applied to damaged skin may result. Osmotic shock or osmotic stress is a sudden change in the solute concentration around a cell, causing a rapid change in the movement of water across its cell membrane. Under conditions of high concentrations of either salts, substrates or any solute in the supernatant, water is drawn out of the cells through osmosis. This also inhibits the transport of substrates and cofactors into the cell thus “shocking” the cell. Alternatively, at low concentrations of solutes, water enters the cell in large amounts, causing it to swell and either burst or undergo apoptosis. Certain of the formulations as described herein can be advantageously employed where it is desirable to minimize osmotic shock.

Viscosity of the compositions can be maintained at the selected level using a pharmaceutically acceptable thickening agent. Suitable viscosity enhancers or thickeners which may be used to prepare a viscous gel or cream with an aqueous base include sodium polyacrylate, xanthan gum, polyvinyl pyrrolidone, acrylic acid polymer, carrageenans, hydroxyethyl cellulose, hydroxypropyl cellulose, methyl cellulose, ethyl cellulose, propyl cellulose, hydroxypropyl methyl cellulose, polyethoxylated polyacrylamides, polyethoxylated acrylates, and polyethoxylated alkane thiols. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents, or by employing a base that has an acceptable level of viscosity.

A pharmaceutically acceptable preservative can be employed to increase the shelf life of the composition. Other suitable preservatives and/or antioxidants for use in liposomal compositions include benzalkonium chloride, benzyl alcohol, phenol, urea, parabens, butylated hydroxytoluene (BHT), butylated hydroxyanisole (BHA), tocopherol, thimerosal, chlorobutanol, or the like, and mixtures thereof, can be employed. If a preservative, such as an antioxidant, is employed, the concentration is typically from about 0.02% to about 2% based on the total weight of the composition, although larger or smaller amounts can be desirable depending upon the agent selected. Reducing agents, as described herein, can be advantageously used to maintain good shelf life of the formulation. It is generally observed that the anhydrous formulations of the embodiments exhibit satisfactory stability, such that a preservative can be omitted from the formulation.

Suitable chelating agents for use in liposomal compositions include ethylene diamine tetraacetic acid, alkali metal salts thereof alkaline earth metal salts thereof, ammonium salts thereof, and tetraalkyl ammonium salts thereof.

The carrier preferably has a pH of between about 4.0 and 10.0, more preferably between about 6.8 and about 7.8. The pH may be controlled using buffer solutions or other pH modifying agents. Suitable pH modifying agents include phosphoric acid and/or phosphate salts, citric acid and/or citrate salts, hydroxide salts (i.e.., calcium hydroxide, sodium hydroxide, potassium hydroxide) and amines, such as triethanolamine. Suitable buffer solutions include a buffer comprising a solution of monopotassium phosphate and dipotassium phosphate, maintaining a pH of between 5.8 and 8; and a buffer comprising a solution of monosodium phosphate and disodium phosphate, maintaining a pH of between 6 and 7.5. Other buffers include citric acid/sodium citrate, and dibasic sodium phosphate/citric acid. The compositions of the embodiments are preferably isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. It can be desirable to include a reducing agent in the formulation, such as vitamin C, vitamin E, or other reducing agents as are known in the pharmaceutical arts.

Surfactants can also be employed as excipients, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

In certain embodiments, it can be advantageous to include additional agents having pharmacological activity. Anti-infective agents include, but are not limited to, anthelmintic (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim. Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine. Anti-inflammatory agents include, but are not limited to, nonsteroidal anti-inflammatory drugs (NSAIDs) such as aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

In some embodiments, liposomal compositions comprise hydrogenated lecithin, C₁₂-C₁₆ alcohols, palmitic acid, or combinations thereof. In some embodiments, hydrogenated lecithin is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, hydrogenated lecithin is provided in a range of about 0.25% to about 10%, about 0.5% to about 8%, about 0.75% to about 6%, or about 1% to about 4% by weight. In some embodiments, hydrogenated lecithin is provided with C₁₂-C₁₆ alcohols, palmitic acid, or combinations thereof. In some embodiments, C₁₂-C₁₆ alcohols are provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, C₁₂-C₁₆ alcohols are provided in a range of about 0.25% to about 10%, about 0.5% to about 8%, about 0.75% to about 6%, or about 1% to about 4% by weight. In some embodiments, palmitic acid is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, palmitic acid is provided in a range of about 0.25% to about 10%, about 0.5% to about 8%, about 0.75% to about 6%, or about 1% to about 4% by weight. In some embodiments, hydrogenated lecithin, C₁₂-C₁₆ alcohols, and palmitic acid are provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, hydrogenated lecithin, C₁₂-C₁₆ alcohols, and palmitic acid are provided in a range of about 0.25% to about 10%, about 0.5% to about 8%, about 0.75% to about 6%, about 1% to about 4%, or about 1% to about 6% by weight. In some embodiments, hydrogenated lecithin, C₁₂-C₁₆ alcohols, and palmitic acid are provided at about 4% by weight. In some embodiments, hydrogenated lecithin, C₁₂-C₁₆ alcohols, and palmitic acid are provided at about 5% by weight.

Liposomal compositions as described herein may further comprise isopropyl palmitate, polyacrylate-13, ascorbyl palmitate, ornithine, ergothioneine, phytosterols, phospholipids, glycolipids, betaine, squalane, polysorbate 20, tocopherol, caprylhydroxamic acid, polyisobutene, ethylhexylglycerin, phenoxyethanol, or combinations thereof. In some embodiments, the isopropyl palmitate, polyacrylate-13, ascorbyl palmitate, ornithine, ergothioneine, phytosterols, phospholipids, glycolipids, betaine, squalane, polysorbate 20, tocopherol, caprylhydroxamic acid, polyisobutene, ethylhexylglycerin, phenoxyethanol, or combinations thereof are provided at least or about 0.0001%, 0.0005%, 0.00055%, 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10%.

Ultraflexible liposomes containing lidocaine solution (˜0.5%), with a membrane containing soybean phosphatidylcholine (SPC) and sodium cholate (NaChol), in a 3.5% w/w carbomer gel were found to be significantly more effective than traditional liposomes or non-liposome formulations. As shown in FIGS. 3A, 3B, and 3C, the cumulative release of drug from the preferred formulation of phospholipid:surfactant of 4:1 is double that of phospholipid alone, and better than the other alternatives tried. This difference is not explained by the remainder of the formulation, see e.g., FIG. 4. The formulation is storage stable for at least 90 days, see FIG. 5. FIG. 6 shows that the onset of action of the preferred formulation is faster than the alternates, and the duration of action is longer, all without generating a peak response significantly greater than LMX4 cream (18.75 mg or 50 mg). Together, these results are unexpected and advantageous. While these effects are demonstrated with lidocaine, it is not believed that the particular drug itself is instrumental to the results, and therefore many other drugs can be delivered using the improved delivery system. For example, see www.drugs.com/drug-class/topical-agents.html: anorectal preparations; antiseptic and germicides; dermatological agents; miscellaneous topical agents; topical acne agents; topical anesthetics; topical anti-infectives; topical anti-rosacea agents; topical antibiotics; topical antifungals; topical antihistamines; topical antineoplastics; topical antipsoriatics; topical antivirals; topical astringents; topical debriding agents; topical depigmenting agents; topical emollients; topical keratolytics; topical non-steroidal anti-inflammatories; topical photochemotherapeutics; topical rubefacient; topical steroids; topical steroids with anti-infectives; mouth and throat products; nasal preparations; nasal antihistamines and decongestants; nasal lubricants and irrigations; nasal steroids; ophthalmic preparations; anti-angiogenic ophthalmic agents; miscellaneous ophthalmic agents; mydriatics; ophthalmic anesthetics; ophthalmic anti-infectives; ophthalmic anti-inflammatory agents; ophthalmic antihistamines and decongestants; ophthalmic diagnostic agents; ophthalmic glaucoma agents; ophthalmic lubricants and irrigations; ophthalmic steroids; ophthalmic steroids with anti-infectives; ophthalmic surgical agents; otic preparations; cerumenolytics; miscellaneous otic agents; otic anesthetics; otic anti-infectives; otic steroids; otic steroids with anti-infectives; sterile irrigating solutions; vaginal preparations; miscellaneous vaginal agents; spermicides; and vaginal anti-infectives.

In addition to lidocaine and the other topical anesthetics discussed herein, agent agents deliverable using the technology are, for example, acetylcholine; acyclovir; alcaftadine; alclometasone; alitretinoin; amcinonide; aminolevulinic acid; androgenic steroids; anthralin; anti-androgenic steroids; antipyrine; apraclonidine; atropine; azelaic acid; azelastine; becaplermin; bepotastine; betamethasone; betaxolol; bexarotene; bimatoprost; brimonidine; brinzolamide; bromfenac; budesonide; calcipotriene; calcitriol; capsaicin; carbachol; carteolol; cetirizine; chloroxylenol; ciclesonide; clobetasol; clocortolone; conjugated estrogens; corticosteroids; crisaborole; cromolyn; cyclopentolate; cyclosporine; desonide; desoximetasone; dexamethasone; dexpanthenol; diclofenac; diflorasone; difluprednate; diphenhydramine; dipivefrin; dorzolamide; doxepin; echothiophate iodide; eflornithine; emedastine; epinastine; epinephrine; estradiol; estrogenic steroids; fluocinolone; fluocinonide; fluorometholone; fluorouracil; flurandrenolide; flurbiprofen; fluticasone; glutathione; glycopyrronium; halcinonide; halebetasol; hydrocortisone; hydroquinone; hydroxyamphetamine; imiquimod; ingenol; iodoquinol; ipratropium; ivermectin; brimonidine; ketoconazole; ketorolac; ketotifen; latanoprost; latanoprostene bunod; levobunolol; levocabastine; lifitegrast; lodoxamide; loteprednol; mechlorethamine; medrysone; menthol; mequinol; methoxsalen; methyl aminolevulinate; methyl salicylate; methylprednisolone; metipranolol; metronidazole; minoxidil; mometasone; mometasone; n-acetyl cysteine; naphazoline; nedocromil; nepafenac; netarsudil; olopatadine; oxymetazoline; pataday; pemirolast; peniciclovir; pheniramine; phenylephrine; physostigmine; pilocarpine; pimecrolimus; polycosanol; pramoxine; prednicarbate; prednisolone; proparacaine; rimexolone; ruxolitinib; suprofen; tacrolimus; tafluprost; tazarotene; tetrahydrozoline; timolol; tirbanibulin; travoprost; tretinoin; triamcinolone; trolamine salicylate; tropicamide; and unoprostone. These include dermal, oral, nasal, otic, aural, vaginal, and anal drugs.

It is therefore an object to provide a topical anesthetic, comprising: liposomes comprising an aqueous inner phase comprising a sodium ion channel blocker; and a lipid bilayer surrounding the aqueous phase, the lipid bilayer comprising lipids having a hydrophilic polar head group and a hydrophobic tail and a surfactant; and a gel supporting the liposomes.

It is a further object to provide a transdermal drug delivery formulation, comprising liposomes comprising an aqueous inner phase comprising a drug; and a lipid bilayer surrounding the aqueous phase, the lipid bilayer comprising lipids having a hydrophilic polar head group and a hydrophobic tail and a surfactant; and a gel supporting the liposomes.

It is a still further object to provide a method topically anesthetizing a subject, comprising administering a pharmaceutically acceptable topical gel formulation comprising liposomes in a hydrogel to a tissue of the subject, the liposomes comprising an aqueous inner phase comprising a sodium ion channel blocker; and a lipid bilayer surrounding the aqueous phase, the lipid bilayer comprising lipids having a hydrophilic polar head group and a hydrophobic tail and a surfactant.

The sodium channel blocker may comprise lidocaine. The sodium channel blocker may be selected from one or more of the group consisting of lidocaine, mepivicaine, prilocaine, bupivacaine, etidocaine, pramoxine, prilocaine, benzocaine, tetracaine, bupivanor, betacaine, topicaine, procaine, chloroprocaine, cocaine, dibucaine, mepivacaine, bupivacaine, levobupivacaine, ropivacaine, articaine, etidocaine, butamben, oxybuprocaine, proxymetacaine, and xylocaine.

The topical anesthetic comprises 0.5-5% w/w, 1-2.5% w/w, or 1.5% w/w of the sodium channel blocker. The topical anesthetic may comprise 1.5% w/w of lidocaine. The liposomes are preferably ultraflexible liposomes. The liposomes may have a diameter of less than 100 nm, less than 90 nm, less than 80 nm, less than 70 nm, or 64.3±2.1 nm.

The liposomes may have a polydispersity index less than 0.1.

The liposomes may have a negative Zeta potential, e.g., less than −10 mV, less than −20 mV, about −21.6 mV, greater than −30 mV, or greater than −25 mV.

The lipids having the hydrophilic polar head group and the hydrophobic tail may comprise phospholipids, phosphatidyl choline, or soy phosphatidyl choline.

The surfactant may comprise a salt of cholic acid, e.g., sodium cholate. The salt of cholic acid is present in a sufficient amount to ensure that the liposomes are ultraflexible liposomes. The salt of cholic acid may comprise sodium cholate present in an amount of at least 5% w/w, at least 10% w/w, at least 15% w/w, in an amount of 20% w/w, less than 25% w/w, less than 30% w/w, less than 35% w/w, or less than 40% w/w.

The gel supporting the liposomes may comprise a carbomer gel, e.g., a 3.5% w/w carbomer gel.

The gel supporting the liposomes may be selected from the group consisting of agar, alginate, alginic acid, arabic gum, gelatin, starch, tragacanth gum, methylcellulose, hydroxyethylcellulose, carboxymethylcellulose, polyacrylic acids, polyacrylate, polymethacrylate, polymethacrylic acid, and polyvinylalcohol.

The topical anesthetic may have a viscosity of less than 7,500 mPa·s, less than 7,000 mPa·s, less than 6,500 mPa·s, or substantially 6,000 mPa·s at a shear rate of 10/s.

The surfactant may be selected from the group consisting of at least one of amide esters, esters with hydroxy-acids, sugar esters, lower alcohol fatty acid esters, esters with propylene glycol, esters with glycerol, triglycerides, mixtures of propylene glycol esters and glycerol esters, mixture of oleic acid esters of propylene glycol and glycerol and polyglycerized fatty acids, non-phosphorous-containing lipids include polyethoxylated fatty acids, PEG-fatty acid diesters, polyethylene glycol glycerol fatty acid esters, alcohol-oil transesterification products, including but not limited to almond oil, arachnidoic acid, behenic acid, capric acid, caprylic acid, caprylic acid diglycerides, caprylic acid mono/diglycerides, caprylic acid monoglycerides, Captex 300, Captex 350, castor oil, coconut oil, corn oil, diacetylated monoglycerides, diglucose monocaprylate, diglucose monocaprylate, ethyl oleate, Gelucire 33/01, glucose monocaprylate, glycerol monolinoleate, glyceryl caprate, glyceryl caprylate, glyceryl caprylate/caprate, glyceryl dilaurate, glyceryl dioleate, glyceryl laurate, glyceryl mono/dioleate, glyceryl monooleate, glyceryl ricinoleate, hydrogenated castor oil, hydrogenated coconut oil, Hydrokote AP5, Imwitor® 375 (glyceryl citrate/lactate/linoleate/oleate), isopropyl myristate, isopropyl palmitate, lauric acid, lauric acid diethanolamide, lauroyl carnitine, lauroyl macrogol-32 2 glycerides, lauryl lactate, linoleic acid, linolenic acid, linoleoyl macrogolglycerides, Miglyol 812, Miglyol 818, mono-and diacetylated monoglycerides, monoacetylated monoglycerides, myristic acid, myristoyl carnitine, oleic acid, palmitoyl carnitine, PEG-10 laurate, PEG-10 oleate, PEG-12 laurate, PEG-12 oleate, PEG-15 oleate, PEG-20 dilaurate, PEG-20 dioleate, PEG-20 distearate, PEG-20 glyceryl laurate, PEG-20 glyceryl oleate, PEG-20 laurate, PEG-20 oleate, PEG-25 trioleate, PEG-30 glyceryl laurate, PEG-30 glyceryl oleate, PEG-32 dilaurate, PEG-32 dioleate, PEG-40 glyceryl laurate, PEG-40 palm kernel oil, PEG-5 oleate, PEG-50 castor oil, PEG-50 hydrogenated castor oil, PEG-6 capric glycerides, PEG-6 caprylic glycerides, PEG-6 caprylic/capric glycerides, PEG-60 almond oil, PEG-60 corn glycerides, PEG-60 hydrogenated castor oil, PEG-8 caprylic/capric glycerides, PEG-8 laurate, PEG-8 oleate, PEG-8 stearate, PEG-9 oleate, PEG-fatty acid mono- and di-ester mixtures, PEGylated glycerol 12-acyloxy-stearate, polyglyceryl oleate, polyglyceryl-10 laurate, polyglyceryl-10 mono, dioleate (Caprol® PEG 860), polyglyceryl-10oleate, polyglyceryl-10 trioleate, polyglyceryl-2 dioleate, Polyglyceryl-3 Polyricinoleate, polyoxyl 35 castor oil, polyoxyl 40 castor oil, propylene glycol dicaprate, propylene glycol dicaprylate, propylene glycol dicaprylate/dicaprate, propylene glycol dioctanoate, propylene glycol monocaprylate, propylene glycol monolaurate, propylene glycol monooleate, propylene glycol ricinoleate, Pureco 100, sodium dioctyl sulfosuccinate, sodium lauryl sarcosinate, sodium lauryl sulfate, sodium stearoyl lactylate, sorbitan monolaurate, sorbitan monooleate sorbitansesquioleate, sorbitan monopalmitate, sorbitan monostearate, sorbitan tristearate, soybean oil, stearic acid, stearoyl macrogol-32 glycerides, stearyl alcohol, sucrose laurate, and combinations thereof.

The topical anesthetic may further comprise dimethyl sulfoxide (DMSO), e.g., 3% DMSO.

It is also an object to provide a topical anesthetic, comprising liposomes comprising: an aqueous inner phase comprising an ion channel blocker; and a membrane surrounding the aqueous inner phase, comprising molecules having hydrophilic polar groups and hydrophobic regions, and a surfactant; and a gel supporting the liposomes.

It is a further object to provide a transdermal drug delivery formulation, comprising: ultraflexible liposomes having a diameter of less than 100 nm, comprising: an aqueous inner phase comprising a drug; and a membrane surrounding the aqueous phase, comprising amphiphilic molecules and a surfactant; and a gel supporting the ultraflexible liposomes.

It is a still further object to provide a method topically anesthetizing a subject, comprising administering to a body surface a pharmaceutically acceptable topical gel formulation comprising liposomes in a hydrogel to a tissue of the subject, the liposomes comprising a membrane comprising amphiphilic molecules and a surfactant surrounding an aqueous inner phase comprising an ion channel blocker.

The ion channel blocker may comprise a sodium ion channel blocker, e.g., lidocaine, mepivicaine, prilocaine, bupivacaine, etidocaine, pramoxine, prilocaine, benzocaine, tetracaine, bupivanor, betacaine, topicaine, procaine, chloroprocaine, cocaine, dibucaine, mepivacaine, bupivacaine, levobupivacaine, ropivacaine, articaine, etidocaine, butamben, oxybuprocaine, proxymetacaine, and xylocaine, in an amount (w/w) of 0.5%-5%, 1-2.5%, or 1.5%.

The membrane may comprise a lipid bilayer surrounding the aqueous phase, the molecules comprising lipids having a hydrophilic polar head group and a hydrophobic tail. The liposomes are preferably ultraflexible liposomes having a diameter of less than 100 nm, or 90 nm, or 80 nm, or 70 nm.

The amphiphilic molecules may comprise phosphatidyl choline.

The surfactant may comprise a salt of cholic acid present in a sufficient amount to ensure that the liposomes in the topical anesthetic are ultraflexible liposomes. Other surfactants may also be employed. The liposomes may further comprise dimethylsulfoxide (DMSO), e.g., 1-5%, 2-4%, or 3% w/w. The surfactant, e.g., salt of cholic acid may comprise sodium cholate. The surfactant may be present in an amount of at least 5% w/w, e.g., 5-50% w/w, 10-40% w/w, or 20-30% w/w.

The amphiphilic molecules comprise phosphatidyl choline and, and the surfactant may comprise 10-40% w/w sodium cholate. The amphiphilic molecules may comprise soy phosphatidyl choline, the surfactant comprise 20% w/w sodium cholate, and the ion channel blocker comprise lidocaine.

The gel may comprise a carbomer gel, agar, alginate, alginic acid, arabic gum, gelatin, starch, tragacanth gum, methylcellulose, hydroxyethylcellulose, carboxymethylcellulose, polyacrylic acids, polyacrylate, polymethacrylate, polymethacrylic acid, and/or polyvinyl alcohol.

The topical anesthetic may have a viscosity of less than 7,000 mPa·s at a shear rate of 10/s.

### BRIEF DESCRIPTION OF THE DRAWINGS

FIG. 1 shows release profiles of ultraflexible and traditional liposomes. The release study was done by introducing the prepared liposomal suspension into a dialysis bag (MWCO 12,000 kDa) and submerging the dialysis bag into a release media containing PBS (pH=7.4) with 20% ethanol in a closed glass vial at 37° C. with a rotation speed at 200 rcf. At predetermined time points, aliquots were withdrawn and analyzed spectrophotometrically at λ=263 nm. The withdrawn samples were replaced with the same volume of fresh media.

FIG. 2 shows percent survival of human keratinocytes treated with ultraflexible (UF) and traditional (TD) liposomes for 24 h. Keratinocytes were seeded in a 96-well plate at a density of 5000 cells/well. 24 hours later, the culture media was replaced with 10 μL of treatment containing either UF or TD liposomes (or PBS as a negative control). After treating the cells for 24 hours, old media with treatment was discarded and fresh media containing MTS solution was added. The UV-Vis absorbance of the cells was read at 490 nm after 2 h of incubation.

FIG. 3A shows permeation profiles through Strat-M membranes of 50 mg of different formulations of liposomal gels.

FIG. 3B shows permeation profiles through Strat-M membranes of lidocaine-containing carbomer gel, LMX4 cream, traditional and ultraflexible liposomal lidocaine gel. In vitro permeation study was performed using a Franz diffusion cell. The receptor compartment consisted of a mixed media containing PBS (pH=7.4) with 20% ethanol. All experiments were carried out for 3 h at 32±0.5° C. with constant stirring at 200 rcf. Before using, the Strat-M membrane was pre-hydrated for 1 h with the release media and then mounted on the top of the receptor chamber of the Franz cell with an effective diffusion area of 1.76 cm². 50 mg of various formulations of lidocaine was applied onto the membrane and aliquot of receptor solution was collected at various time points and replaced with the same volume of release media. The lidocaine concentration was read spectrophotometrically at λ=263 nm.

FIG. 3C shows permeation profiles through Strat-M membranes of 200 mg lidocaine-containing carbomer gel, LMX4 cream, traditional and ultraflexible liposomal lidocaine gel, and Alocane gel.

FIG. 4 shows the viscosity of LMX4 cream, Alocane gel, UFL and TL gels at 32° C.

FIG. 5 shows stability of ultraflexible liposomal lidocaine gel over 3 months storage at 4° C.

FIG. 6 shows the anesthetic effect of carbomer gel, traditional liposomal lidocaine gel, ultraflexible liposomal lidocaine gel and LMX4 cream on the latency of tail flick after 50 mg lidocaine equivalent application. 25 male Sprague Dawley rats weighing 290±25 g were distributed into five groups (n=5). To carry out the experiment, a specified area of rat tail was exposed to a radiant heat source, while the rats were loosely fixed. Baseline latency for each rat was detected, which was defined as the average time of four measurements before the application of any formulation. As flicking of rat tail occurred, the required time was recorded. Tail flick latency time was determined as the time from the application of the heat exposure to the flicking of the tail.

### DETAILED DESCRIPTION OF THE PREFERRED EMBODIMENTS

This disclosure is intended to be interpreted to encompass all combinations and permutations of the disclosed compositions. The disclosure is also intended to be interpreted to encompass specific components and compositions limited to the preferred embodiments (without additional components or substantially without additional components). The disclosure is further intended to encompass inclusion all of the cited prior art as improved by the teachings hereof.

In general, the invention provides a liposome having an ultraflexible membrane, supported in a gel. The ultraflexible liposome contains an aqueous phase which includes a pharmaceutical agent. The formulation is effective topically to release the pharmaceutical agent over a period of time.

The ultraflexible or traditional liposomes were prepared by film hydration technique by mixing lidocaine and soybean phosphatidylcholine (SPC) with or without the detergent, sodium cholate (NaChol). The prepared liposomes were evaluated for the particle size (Zetasizer), toxicity (MTS assay) and in vitro release (dialysis). The liposomal lidocaine gel was prepared by mixing liposomes suspensions with 3.5% w/w carbomer gel. The formulated gels were evaluated for pH, viscosity, strat-M (a skin-like membrane) permeation. The analgesic effect of the prepared formulations was tested and compared to commercially available LMX4 lidocaine cream (a commercially available lipid-based lidocaine cream) using a tail flick test.

### Methods

Preparation of lidocaine-loaded (ultraflexible) liposomes by thin-film hydration method. Lidocaine-loaded liposomal formulations were prepared by the thin-film hydration method. 50 mg Soybean phosphatidylcholine (SPC), 20 mg lidocaine, and different amount (0, 10, 20, 30 and 50%, w/w) of sodium cholate (NaChol) were dissolved in 2 mL methanol:chloroform mixture at 1:1 v/v ratio. Using a rotary evaporator, the thin lipid film on the internal surface of the round-bottomed flask was formed. Then the film was hydrated with 1 mL of PBS (pH=7.4) followed by 5 min sonication and vortex. The resulting liposomal suspension was extruded through 200 nm polycarbonate membranes for particle homogenization. A different formulation (35 mg SPC, 15 mg cholesterol, or Chol with 20% w/w NaChol) was prepared using the same method)

Characterization of lidocaine-loaded UFLs and TLs: determination of the particle size and zeta potential. Aliquots of UFLs or TLs dispersion (150 μL) were diluted with deionized water (850 μL) to examine the zeta potential and the particle size of the liposomes. The zeta potential and particle size were measured by the method of dynamic light scattering using Malvern Zetasizer (Malvern Instruments Corp; Nano ZS ZEN 3600, Worcestershire, UK).

In vitro release study. The in vitro release study was performed using the dialysis method. Briefly, an equivalent amount of 1 mL lidocaine-loaded UFL or TL dispersion was introduced into dialysis bags with a molecular weight cutoff 12,000 kDa. The dialysis bags were suspended in a release media containing PBS and 20% ethanol at 37° C. at a speed of rotation 200 rcf and placed within a closed glass vial. The samples (500 μL) were withdrawn and analyzed spectrophotometrically at λ=263 nm at predetermined time points. The withdrawn samples were replaced with the same volume of PBS.

Cytotoxicity assays. Human keratinocytes were cultured to indicate the cytotoxicity of the UFLs and TLs prepared. Keratinocytes were seeded in a 96-well plate at a density of 5000 cells/well. 24 hours later, the culture media was replaced with 10 μL of treatment containing either UFLs or TLs (or PBS as a negative control). After treating the cells for 24 hours, old media with treatment was discarded and fresh media containing MTS solution was added. The UV-Vis absorbance of the cells was read at 490 nm after 2 h of incubation.

Preparation of liposomal lidocaine gel. To prepare carbomer gel, 3.5 mg Carbopol 940 was added in 10 mL distilled water and stirred at 150 rcf in a refrigerator. At least 12 h later, triethanolamine was added slowly to the carbomer gel until the pH reaches 7. Then the carbomer gel was mixed via probe sonication at an amplitude of 20 MHz for 10 s and was then vortexed for 1 min. To prepare liposomal lidocaine gel, liposomal suspension (or lidocaine solution) and carbomer gel at 3:1 v/v ratio was mixed by vortexing and stirring for 5 min. For a follow-up experiment where DMSO was tested as a chemical enhancer, 3% DMSO was added during the mixing of liposomal suspension and carbomer gel.

Permeation studies. Transdermal diffusion tests were conducted using a Franz cell and Millipore Strat-M membranes, a type of membrane commonly used in predictive studies for diffusion in human skin. The Franz diffusion cells were loaded with 50 mg samples (or 18.75 mg LMX4 cream to account for the same amount of lidocaine in liposomal lidocaine gels). The receptor compartment consisted of a mixed media containing PBS (pH=7.4) with 20% ethanol. All experiments were carried out for 3 h at 32±0.5° C. with constant stirring at 200 rcf. Before using, the Strat-M membrane was pre-hydrated for 1 h with the release media and then mounted on the top of the receptor chamber of the Franz cell with an effective diffusion area of 1.76 cm². 50 mg of various formulations of lidocaine was applied onto the membrane and spread evenly to cover all the exposed area of the membrane. Aliquots of receptor solution were collected at various time points and replaced with the same volume of release media. The lidocaine concentration was read spectrophotometrically at λ=263 nm. The permeation studies were also performed by loading 200 mg samples onto the membrane using the same technique. All determinations were accomplished in triplicate, and the results were represented as the mean±SD. Due to the findings in the permeation studies, ultraflexible liposomal formulation prepared by SPC and 20% w/w NaChol was chosen for further examinations. In the following text, the term “ultraflexible” refers to this formulation if there is no specific explanation.

Viscosity tests. The viscosities of the formulations were determined using a micro VISC from RheoSense (San Ramon, CA, USA) at skin temperature 32° C. Viscosity assays were performed for ultraflexible and traditional liposomal lidocaine gels and two commercially available products, Alocane gel and LMX4 cream. Using the provided pipette for the instrument, 10 μL of sample were injected into the viscometer for each viscosity measurement, and the viscosity was measured at shear rates of 10 to 60 s−1 at 32° C. Samples were run in triplicate.

Stability of ultraflexible liposomal lidocaine gels. To examine the leakage of lidocaine from the UFL gel throughout storage, physical stability studies were conducted. The prepared UFL dispersion was stored in tightly sealed glass vials at 4±1° C. for 3 months. Lidocaine content in the stored gel formulations was determined periodically. Any change in the appearance of the stored gel formulations was recorded.

Tail-flick test. 30 male Sprague Dawley rats weighing 290±25 g were distributed into five groups (n=5) with 5 extra rats. All rats were kept at specified environmental conditions (temperature: 20±2° C., humidity: 40±10% RH) while conducting the experiment. The rats were accommodated to the specified environmental conditions for 10 days before carrying out tail flick test. To carry out the experiment, a specified area of rat tail was exposed to a radiant heat source, while the rats were loosely fixed. Baseline latency for each rat was detected, which was defined as the mean time of three measurements before application of any formulation. The rats whose baseline latency average time was more or less than the time range 6-8 seconds were excluded from the experiment. As flicking of rat tail occurred, the required time was recorded. A 20-second cutoff time was applied to keep away from tissue thermal injury. Tail flick latency time was determined as the time from the application of the heat exposure to the flicking of the tail. Five groups of the rats were handled gently. These groups of the rats were treated with 50 mg carbomer gel (first group), 50 mg ultraflexible liposomal lidocaine gel (second group), 50 mg traditional liposomal lidocaine gel, 50 mg LMX4 cream (fourth group), and 18.75 mg LMX4 cream (fifth group). 18.75 mg LMX4 cream was used to account for the same amount of lidocaine in the liposomal lidocaine gel groups. The time course of lidocaine response of individual formulation was represented by plotting the mean of latency times as a function of time.

### Results

Characterization of UFLs and TLs. The Zetasizer data of UFLs (prepared by SPC with 20% w/w NaChol) and TLs (Table 1) showed that both formulations had uniform size distribution, while NaChol in the UFL formulation resulted in a smaller average size and a negative surface charge.

TABLE 1
The size, PDI, and zeta potential of ultraflexible
and traditional liposomes.

| | | | Zeta |
|---|---|---|---|
| Formulations | Size (nm) | PDI | potential (mV) |
| SPC/NaChol/Lidocaine | 64.3 ± 2.1 | 0.08 ± 0.01 | −21.6 ± 1.2 |
| (Ultraflexible liposomes) | | | |
| SPC/Lidocaine | 139.3 ± 1.8 | 0.09 ± 0.01 | 0 |
| (Traditional liposomes) | | | |

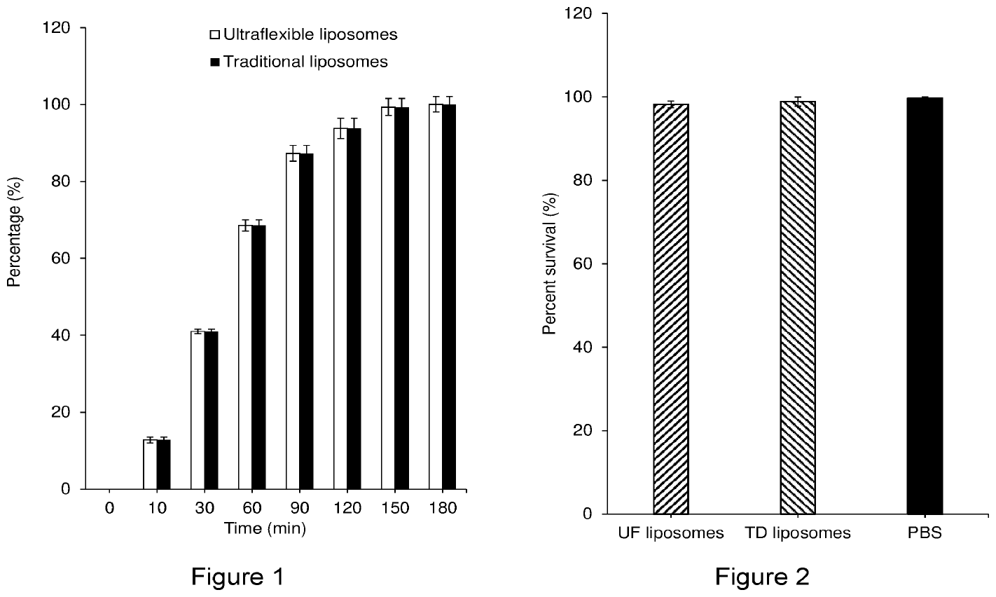

UFLs and TLs exhibited similar in vitro release profiles. The release profiles of lidocaine from the prepared UFLs (prepared by SPC with 20% w/w NaChol) and TLs are represented in FIG. 1. More than 80% of the drug content of the prepared UFLs as well as TLs was released after 90 min. Statistical examination proved that there is no significant difference in the release values of lidocaine from these two formulations (P>0.05). Both liposomes released their drug contents according to a first-order kinetic model (R²>0.95). The controlled release of lidocaine from UFLs and TLs shown in FIG. 1 was expected due to the role of liposomes as a drug reservoir for prolonged release.

UFLs and TLs showed good biocompatibility. A colorimetric MTS assay was performed to test the cytotoxicity of these liposomal formulations in human keratinocytes. The cells were treated with 10 μL non-diluted UFL (prepared by SPC with 20% w.w NaChol) or TL suspension plus 90 μL culture media. As shown in FIG. 2, the percent survival rates of these liposomes were both greater than 98%, indicating the great biocompatibility of SPC liposomes.

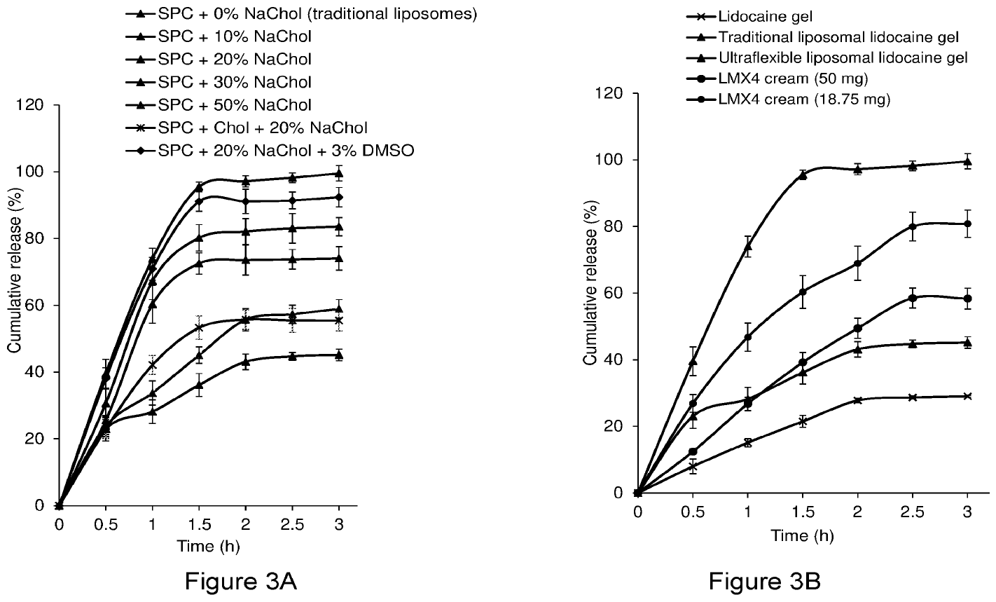

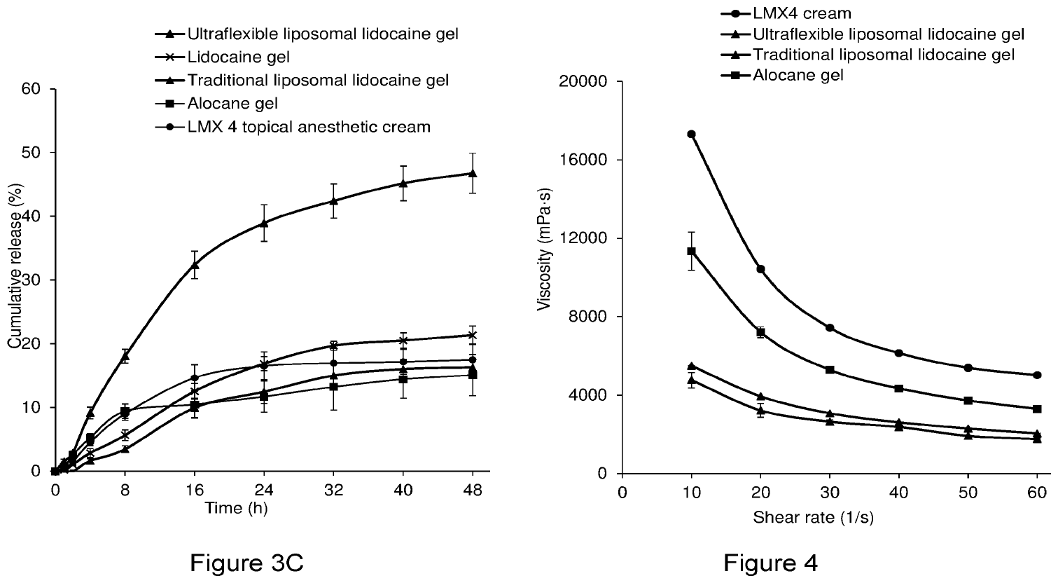

SPC with 20% NaChol demonstrated optimal penetration ability. A transdermal diffusion study was conducted in a Franz cell to test their skin permeation ability. First, the penetration ability of different formulations of liposomes with various percentages of the surfactant was tested, NaChol. It was shown in FIG. 3A that the amount of lidocaine penetrated through the Strat-M membrane (which mimics real skin) increased when NaChol increased from 0 to 20% w/w and decreased when NaChol kept increasing from 20% to 50%. Having determined 20% w/w NaChol was the ideal amount of surfactant for SPC liposomes w/w, cholesterol was added when preparing for the liposomes or including DMSO during liposomal gel formation to see if cholesterol or DMSO would furthermore enhance the drug penetration. Unfortunately, neither of these two factors demonstrated a better release profile than liposomes made from SPC with 20% w/w NaChol. Therefore, this optimal formulation was compared with plain lidocaine gel and LMX4 cream (FIG. 3B). Almost all the lidocaine (95.52%) encapsulated in UF liposomal lidocaine gel penetrated through the skin within the first 1.5 h, while the traditional liposomal lidocaine gel and LMX4 cream led to 36.17% and 39.2% cumulative release of lidocaine, respectively. Notably, since the gel contains 1.5% w/w lidocaine while LMX4 contains 4% w/w lidocaine, a release study was conducted with only 18.75 mg LMX4 cream to account for the same amount of lidocaine in the other groups, and a better release profile compared to 50 mg LMX4 cream was presented. This is probably caused by the heavy and thick texture of LMX4 cream which might clog the membrane after a certain amount of cream enters it. Then, to better explore this possibility, another permeation study with 200 mg sample on the membrane (FIG. 3C) was conducted. As expected, the release profile plateau time extended from 2.5 h to 40 h. Besides, all the formulations demonstrated at least 50% reduction in the amount of drug permeated through the membrane. This further confirmed that the amount of sample for a specific area of application exists a critical value. Any amount beyond that value will lead to a less efficient outcome. Overall, from these permeation studies, it can be concluded that adding 20% w/w NaChol in liposomal formulation greatly improved skin penetration ability, making the ultraflexible liposomal vehicle an effective tool in transdermal drug delivery. It is presumed that SPC plus 40% w/w NaChol has intermediate properties between the 30% w/w and 50% w/w, versions, and may represent an upper bound to achieve 65% release. Similarly, 10% w/w NaChol also represents a lower bound based on available data to achieve 65% release, though data for 5% NaChol are not shown, and may also exceed 65%.

Viscosity of UFL and TL gel. Due to the result from permeation studies, ultraflexible liposomes prepared using SPC with 20% w/w NaChol were chosen for further studies and termed “ultraflexible”. The viscosity of LMX4 cream, Alocane gel, UFL and TL gels was examined at 32° C. to mimic skin surface temperature. As indicated in FIG. 4, even though liposomal lidocaine gel groups showed a lower viscosity compared to the other two commercial products, they both followed the same trends that as sheer rate increased, their viscosity decreased. These data proved that the liposomal gel formulations would be appropriate for spreading and staying on the skin.

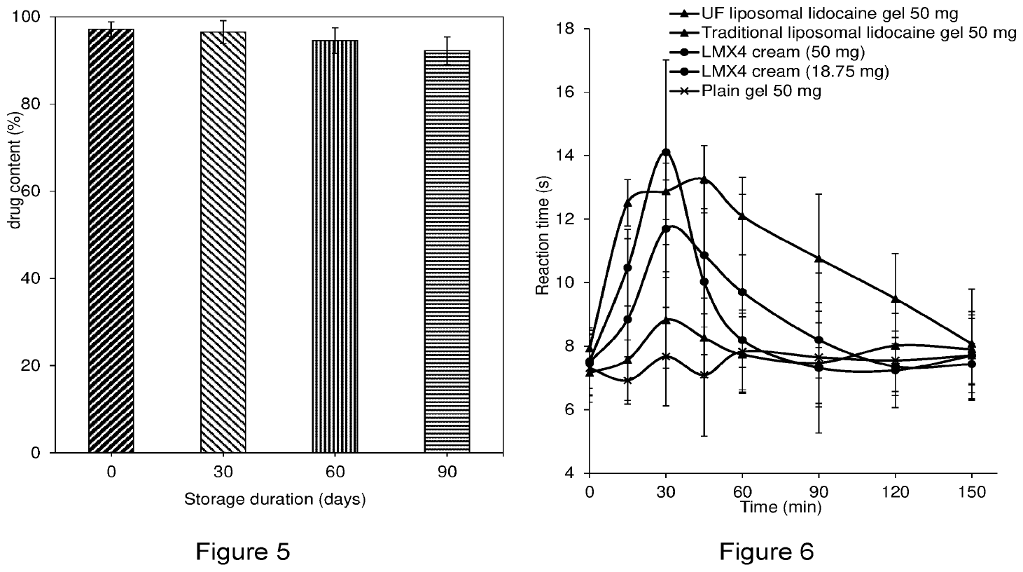

Ultraflexible liposomal lidocaine gel showed good stability over 3 months storage at 4° C. The Stability study of the ultraflexible liposomal lidocaine gel showed no significant decrease in drug content over 3 months for the stored formulations at 4° C. (FIG. 5).

Ultraflexible liposomal lidocaine gel exhibited an enhanced local anesthetic effect. Tail flick test is a test of the pain response in animals used in basic pain research and to measure the effectiveness of analgesics by observing the reaction to heat. These rodents' tails will be given different topical lidocaine products, which are responsible for weakening the response to pain. Under these weakened responses to pain, the effectiveness of the drugs can be tested by exposing the tail to constant heat and measuring how long it takes to flick, signaling its response to the pain. FIG. 6 shows the reaction time of the rats to flip their tails away from the heat stimulus after the application of different lidocaine-containing formulations or plain carbomer gel (negative control). An increased reaction time was observed in both UFL lidocaine gel and LMX4 groups after applying the formulations on the tails for 15 min. However, the analgesic effect plateaued after 30 min application for the 50 mg LMX4 group while the UF group maintained a prolonged effect after 2 hours. This difference can also be noted in the values of the area under curve (AUC). The maximum AUC_{0-150 min} value was found to be 1639.97±201.64 seconds minutes (s·min) for the UFL lidocaine gel group, which is 1.5-folds more than the value of the control group (1127.79±206.61 s·min), 1.25-folds more than the 50 mg LMX4 group (1311.86±224.41 s·min), 1.23-folds more than the 18.75 mg LMX4 group (1323.75±272.2 s·min) and 1.39-folds more than traditional liposomal group (1180.76±127.76 s·min). According to the above results, UFL formulations of lidocaine in gel preparations played an important part in improving the local anesthetic activity of the lidocaine. This can be explained by the negative charge of UFL lidocaine gel. The electrostatic repulsion generated between UFLs and intercellular skin components results in rapid penetration of negatively charged UFLs through follicles of different skin layers. The lipoidal character of UFLs also aids in enhanced skin permeation of lidocaine which is beneficial to localized anesthetic efficacy.

Conclusion. Liposomes are one of the most common drug carriers used in a variety of fields. Embedding the liposomal suspension in gel dosage form not only provides the possibility of localized skin application but also increases the stability of liposomes. With the help of the detergent in ultraflexible liposomal lidocaine gel, an improved skin permeation, as well as enhanced local anesthetic effect is achieved.

Compared to the commercially available lipid-based lidocaine cream, LMX4, ultraflexible liposomes possess a better in vitro skin penetration profile and enhanced anesthetic ability shown in the animal tail-flick study. Considering LMX4 has been reported to be an effective analgesia for newborn circumcision, and the most preferred topical anesthetic for dermatological laser and skin microneedling, it can be concluded that the ultraflexible liposomes may be useful for the development of an effective alternative to painful lidocaine injections in clinical settings.

All publications and patents mentioned in this specification are herein expressly incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A topical drug formulation, comprising:
liposomes comprising:
an aqueous inner phase comprising a drug; and
a membrane surrounding the aqueous inner phase, comprising molecules having hydrophilic polar groups and hydrophobic regions, and a surfactant; and
a gel supporting the liposomes.

2. The topical drug formulation according to claim 1, wherein:
the drug comprises an ion channel blocker; and
the membrane comprises a lipid bilayer surrounding the aqueous phase, the molecules comprising lipids having a hydrophilic polar head group and a hydrophobic tail.

3. The topical drug formulation according to claim 2, wherein the ion channel blocker comprises lidocaine.

4. The topical drug formulation according to claim 2, wherein the ion channel blocker is selected from one or more of the group consisting of mepivicaine, prilocaine, bupivacaine, etidocaine, pramoxine, prilocaine, benzocaine, tetracaine, bupivanor, betacaine, topicaine, procaine, chloroprocaine, cocaine, dibucaine, mepivacaine, bupivacaine, levobupivacaine, ropivacaine, articaine, etidocaine, butamben, oxybuprocaine, proxymetacaine, and xylocaine.

5. The topical drug formulation according to claim 2, wherein the topical anesthetic comprises 1-2.5% w/w of the ion channel blocker.

6. (canceled)

7. The topical drug formulation according to claim 1, wherein the liposomes are ultraflexible liposomes having a diameter of less than 80 nm.

8. The topical drug formulation according to claim 1, wherein the liposomes have a polydispersity index less than 0.1.

9. The topical drug formulation according to claim 1, wherein the molecules comprise phosphatidyl choline.

10. The topical drug formulation according to claim 1, wherein the surfactant comprises a salt of cholic acid present in a sufficient amount to ensure that the liposomes in the topical anesthetic are ultraflexible liposomes.

11. The topical drug formulation according to claim 10, wherein the salt of cholic acid comprises sodium cholate present in an amount of at least 5% w/w, the molecules comprise phosphatidyl choline, the drug comprises an ion channel blocker, the liposomes are ultraflexible liposomes having a diameter of less than 80 nm, and the gel comprises a carbomer gel.

12. The topical drug formulation according to claim 1, wherein the molecules comprise phosphatidyl choline and, and the surfactant comprises 10-40% w/w sodium cholate.

13. The topical drug formulation according to claim 1, wherein the molecules comprise soy phosphatidyl choline, the surfactant comprises 20% w/w sodium cholate, and the ion channel blocker drug comprises lidocaine.

14. The topical drug formulation according to claim 1, wherein the gel comprises a carbomer gel.

15. The topical drug formulation according to claim 1, wherein the gel is selected from the group consisting of agar, alginate, alginic acid, arabic gum, gelatin, starch, tragacanth gum, methylcellulose, hydroxyethylcellulose, carboxymethylcellulose, polyacrylic acids, polyacrylate, polymethacrylate, polymethacrylic acid, and polyvinyl alcohol.

16. The topical drug formulation according to claim 1, wherein the topical anesthetic has a viscosity of less than 7,000 mPa·s at a shear rate of 10/s.

17. The topical drug formulation according to claim 1, wherein the surfactant is selected from the group consisting of at least one of sodium dioctyl sulfosuccinate, sodium lauryl sulfate, lauric acid diethanolamide, sodium lauryl sarcosinate, lauroyl carnitine, palmitoyl carnitine and myristoyl carnitine, sodium stearoyl lactylate, lauryl lactate, glucose monocaprylate, diglucose monocaprylate, sucrose laurate, sorbitan monolaurate, sorbitan monopalmitate, sorbitan monooleate sorbitansesquioleate, sorbitan monostearate and sorbitan tristearate, ethyl oleate, isopropyl myristate and isopropyl palmitate, propylene glycol monolaurate, propylene glycol ricinoleate, propylene glycol monooleate, propylene glycol monocaprylate, propylene glycol dicaprylate, propylene glycol dicaprate, propylene glycol dioctanoate, glyceryl monooleate, glyceryl ricinoleate, glyceryl laurate, glyceryl dilaurate, glyceryl dioleate, glycerol monolinoleate, glyceryl monooleate, glyceryl dioleate, glyceryl caprylate, glyceryl caprate, caprylic acid monoglycerides, caprylic acid diglycerides, monoacetylated monoglycerides, diacetylated monoglycerides, polyglyceryl-2 dioleate, polyglyceryl-10 trioleate, polyglyceryl-10 laurate, polyglyceryl-10 oleate, and polyglyceryl-10 mono, dioleate, PEG-8 laurate, PEG-8 oleate, PEG-8 stearate, PEG-9 oleate, PEG-10 laurate, PEG-5 oleate, PEG-10 oleate, PEG-12 laurate, PEG-12 oleate, PEG-15 oleate, PEG-20 laurate, PEG-20 oleate, PEG-20 dilaurate, PEG-20 dioleate, PEG-20 distearate, PEG-32 dilaurate, PEG-32 dioleate, PEGylated glycerol 12-acyloxy-stearate, PEG-20 glyceryl laurate, PEG-30 glyceryl laurate, PEG-40 glyceryl laurate, PEG-20 glyceryl oleate and PEG-30 glyceryl oleate, polyoxyl 40 castor oil, polyoxyl 35 castor oil, PEG-25 trioleate, PEG-60 corn glycerides, PEG-60 almond oil, PEG-40 palm kernel oil, PEG-50 castor oil, PEG-50 hydrogenated castor oil, PEG-60 hydrogenated castor oil, PEG-8 caprylic/capric glycerides, lauroyl macrogol-32 glycerides, linoleoyl macrogolglycerides, stearoyl macrogol-32 glycerides, PEG-6 caprylic glycerides, PEG-6 capric glycerides, Polyglyceryl-3 Polyricinoleate and ethyl oleate.

18. The topical drug formulation according to claim 1, further comprising dimethyl sulfoxide.

19. A transdermal drug delivery formulation, comprising:
ultraflexible liposomes having a diameter of less than 100 nm, comprising:
an aqueous inner phase comprising a drug; and
a membrane surrounding the aqueous phase, comprising amphiphilic molecules and a surfactant; and
a gel supporting the ultraflexible liposomes.

20-34. (canceled)

35. The topical drug formulation according to claim 11, wherein the liposomes have a Zeta potential lower than −10 mV.

36-62. (canceled)

63. A method of topically anesthetizing a subject, comprising administering a pharmaceutically acceptable topical gel formulation comprising ultraflexible liposomes in a hydrogel to a tissue of the subject, the ultraflexible liposomes comprising:
an aqueous inner phase comprising a sodium ion channel blocker; and
a lipid bilayer surrounding the aqueous phase, the lipid bilayer comprising lipids having a hydrophilic polar head group and a hydrophobic tail and a surfactant,
wherein at least 80% of the sodium ion channel blocker is released within 90 minutes with first order release kinetics.
